SEQUENCE LISTING (14 sequences)

SEQ ID NO: 1 - protein, 303 aa, Hepatitis C virus
  MISC_FEATURE (1)..(303): HCV E2 polypeptide tr|C4MR37|384-746
YNKFNSSGCSERLASCRPLTDFAQGWGPISYANGSGPDQRPYCWHYPPRPCGIVPAKSVCGPVYCFTPSPVVVGTTDRSGAPTYSWGENETDVFILNNTRPPLGNWFGCTWMNSSGFTKVCGAPPCVIGGVGNNTLRCPTDCFRKHPEATYSRCGSGPWITPRCLVNYPYRLWHYPCTINYTIFKVRMYVGGVEHRLEAACNWTRGERCDLEDRDRSELSPLLLSTTQWQVLPCSFTTLPALSTGLIHLHQNIVDVQYLYGVGSSIASWAIKWDYVVLLFLLLADARVCSCLWMMLLISQAEA

SEQ ID NO: 2 - protein, 236 aa, Homo sapiens
  MISC_FEATURE (1)..(236): CD81 AMINO ACID SEQUENCE NP_004347.1
MGVEGCTKCIKYLLFVFNFVFWLAGGVILGVALWLRHDPQTTNLLYLELGDKPAPNTFYVGIYILIAVGAVMMFVGFLGCYGAIQESQCLLGTFFTCLVILFACEVAAGIWGFVNKDQIAKDVKQFYDQALQQAVVDDDANNAKAVVKTFHETLDCCGSSTLTALTTSVLKNNLCPSGSNIISNLFKEDCHQKIDDLFSGKLYLIGIAAIVVAVIMIFEMILSMVLCCGIRNSSVY

SEQ ID NO: 3 - protein, 90 aa, Homo sapiens
  MISC_FEATURE (1)..(90): CD81 Chain A amino acid sequence. Re Crystal Structure Of Human CD81 Extracellular Domain 1G8Q_A  GI13399775
FVNKDQIAKDVKQFYDQALQQAVVDDDANNAKAVVKTFHETLDCCGSSTLTALTTSVLKNNLCPSGSNIISNLFKEDCHQKIDDLFSGKH

SEQ ID NO: 4 - protein, 90 aa, Homo sapiens; identical to SEQ ID NO: 3
  MISC_FEATURE (1)..(90): CD81 Chain B amino acid sequence.  Re Crystal Structure Of Human CD81 Extracellular Domain 1G8Q_B  GI13399776.

SEQ ID NO: 5 - protein, 363 aa, Hepatitis C virus
  MISC_FEATURE (1)..(363): P26664[384-746], Genome polyprotein, Hepatitis C virus genotype 1a (isolate 1)
ETHVTGGSAGHTVSGFVSLLAPGAKQNVQLINTNGSWHLNSTALNCNDSLNTGWLAGLFYHHKFNSSGCPERLASCRPLTDFDQGWGPISYANGSGPDQRPYCWHYPPKPCGIVPAKSVCGPVYCFTPSPVVVGTTDRSGAPTYSWGENDTDVFVLNNTRPPLGNWFGCTWMNSTGFTKVCGAPPCVIGGAGNNTLHCPTDCFRKHPDATYSRCGSGPWITPRCLVDYPYRLWHYPCTINYTIFKIRMYVGGVEHRLEAACNWTRGERCDLEDRDRSELSPLLLTTTQWQVLPCSFTTLPALSTGLIHLHQNIVDVQYLYGVGSSIASWAIKWEYVVLLFLLLADARVCSCLWMMLLISQAEA

SEQ ID NO: 6 - protein, 363 aa, Hepatitis C virus
  MISC_FEATURE (1)..(363): Q9WMX2[384-746], Genome polyprotein, Hepatitis C virus genotype 1b (isolate Con1)
GTYVTGGTMAKNTLGITSLFSPGSSQKIQLVNTNGSWHINRTALNCNDSLNTGFLAALFYVHKFNSSGCPERMASCSPIDAFAQGWGPITYNESHSSDQRPYCWHYAPRPCGIVPAAQVCGPVYCFTPSPVVVGTTDRFGVPTYSWGENETDVLLLNNTRPPQGNWFGCTWMNSTGFTKTCGGPPCNIGGIGNKTLTCPTDCFRKHPEATYTKCGSGPWLTPRCLVHYPYRLWHYPCTVNFTIFKVRMYVGGVEHRLEAACNWTRGERCNLEDRDRSELSPLLLSTTEWQVLPCSFTTLPALSTGLIHLHQNVVDVQYLYGIGSAVVSFAIKWEYVLLLFLLLADARVCACLWMMLLIAQAEA

SEQ ID NO: 7 - protein, 367 aa, Hepatitis C virus
  MISC_FEATURE (1)..(367): P26660[384-750], Genome polyprotein, Hepatitis C virus genotype 2a (isolate HC-J6)
QTHTVGGSTAHNARTLTGMFSLGARQKIQLINTNGSWHINRTALNCNDSLHTGFLASLFYTHSFNSSGCPERMSACRSIEAFRVGWGALQYEDNVTNPEDMRPYCWHYPPRQCGVVSASSVCGPVYCFTPSPVVVGTTDRLGAPTYTWGENETDVFLLNSTRPPQGSWFGCTWMNSTGYTKTCGAPPCRIRADFNASMDLLCPTDCFRKHPDTTYIKCGSGPWLTPRCLIDYPYRLWHYPCTVNYTIFKIRMYVGGVEHRLTAACNFTRGDRCNLEDRDRSQLSPLLHSTTEWAILPCTYSDLPALSTGLLHLHQNIVDVQFMYGLSPALTKYIVRWEWVVLLFLLLADARVCACLWMLILLGQAEA

SEQ ID NO: 8 - protein, 369 aa, Hepatitis C virus
  MISC_FEATURE (1)..(369): Q81495[384-752], Genome polyprotein, Hepatitis C virus genotype 3a (isolate k3a)
STHVTAGQAARNAYGITSLFSVGAKQNLQLINTNGSWHINRTALNCNESINTGFIAGLFYYHKFNSTGCPQRLSSCKPITFFKQGWGPLTDANITGPSDDKPYCWHYAPRPCGIVPALNVCGPVYCFTPSPVVVGTTDAKGAPTYTWGANKTDVFLLESLRPPSGRWFGCTWMNSTGFVKTCGAPPCNIYGDGRDAQNESDLFCPTDCFRKHPEATYSRCGAGPWLTPRCLVDYPYRLWHYPCTVNFTLFKVRMFVGGFEHRFTAACNWTRGERCDIEDRDRSEQHPLLHSTTELAILPCSFTPMPALSTGLIHLHQNIVDVQYLYGIGSGMVGWALKWEFVILIFLLLADARVCVALWLILTISQAEA

SEQ ID NO: 9 - protein, 370 aa, Hepatitis C virus
  MISC_FEATURE (1)..(370): Q81487[385-754], Genome polyprotein, Hepatitis C virus genotype 3b (isolate Tr-Kj)
TTHTTGGSAAQATAGFTSFFTRGPSQNLQLVNSNGSWHINSTALNCNDSLNTGFIAGLFYYHKFNSSGCPERMSSCKPITYFNQGWGPLTDANINGPSEDRPYCWHYPPRPCNITKPLNVCGPVYCFTPSPVVVGTTDIKGLPTYRFGVNESDVFLLTSLRPPQGRWFGCVWMNSTGFVKTCGAPPCNIYGGMKDIEANQTHLKCPTDCFRKHHDATFTRCGSGPWLTPRCLVDYPYRLWHYPCTVNFSIFKVRMFVGGHEHRFSAACNWTRGERCDLEDRDRSEQQPLLHSTTDSLILPCSFTPMRRLSTGLIHLHQNIVDVQYLYGVGSAVVGWALKWEFVVLVFLLLADARVCVALWMMLLISQAEA

SEQ ID NO: 10 - protein, 367 aa, Hepatitis C virus
  MISC_FEATURE (1)..(367): P26661[384-750], Genome polyprotein, Hepatitis C virus genotype 2b (isolate HC-J8)
TTYSSGQEAGRTVAGFAGLFTTGAKQNLYLINTNGSWHINRTALNCNDSLQTGFLASLFYTHKFNSSGCPERLSSCRGLDDFRIGWGTLEYETNVTNDGDMRPYCWHYPPRPCGIVPARTVCGPVYCFTPSPVVVGTTDKQGVPTYTWGENETDVFLLNSTRPPRGAWFGCTWMNGTGFTKTCGAPPCRIRKDYNSTIDLLCPTDCFRKHPDATYLKCGAGPWLTPRCLVDYPYRLWHYPCTVNFTIFKARMYVGGVEHRFSAACNFTRGDRCRLEDRDRGQQSPLLHSTTEWAVLPCSFSDLPALSTGLLHLHQNIVDVQYLYGLSPALTRYIVKWEWVILLFLLLADARICACLWMLIILGQAEA

SEQ ID NO: 11 - protein, 363 aa, Hepatitis C virus
  MISC_FEATURE (1)..(363): O39929[384-746], Genome polyprotein, Hepatitis C virus genotype 4a (isolate ED43)
ETHVSGAAVGRSTAGLANLFSSGSKQNLQLINSNGSWHINRTALNCNDSLNTGFLASLFYTHKFNSSGCSERLACCKSLDSYGQGWGPLGVANISGSSDDRPYCWHYAPRPCGIVPASSVCGPVYCFTPSPVVVGTTDHVGVPTYTWGENETDVFLLNSTRPPHGAWFGCVWMNSTGFTKTCGAPPCEVNTNNGTWHCPTDCFRKHPETTYAKCGSGPWITPRCLIDYPYRLWHFPCTANFSVFNIRTFVGGIEHRMQAACNWTRGEVCGLEHRDRVELSPLLLTTTAWQILPCSFTTLPALSTGLIHLHQNIVDVQYLYGVGSAVVSWALKWEYVVLAFLLLADARVSAYLWMMFMVSQVEA

SEQ ID NO: 12 - protein, 364 aa, Hepatitis C virus
  MISC_FEATURE: O39928[384-747], Genome polyprotein, Hepatitis C virus genotype 5a (isolate EUH1480)
RTHTVGGTVGQGLKSLTSFFNPGPQRQLQFVNTNGSWHINSTALNCNDSLQTGFIAGLMYAHKFNSSGCPERMSSCRPLAAFDQGWGTISYATISGPSDDKPYCWHYPPRPCGVVPARDVCGPVYCFTPSPVVVGTTDRRGCPTYNWGSNETDILLLNNIRPPAGNWFGCTWMNSTGFVKNCGAPPCNLGPTGNNSLKCPTDCFRKHPDATYTRCGSGPWLTPRCLVHYPYRLWHYPCTVNYTIFKVRMFIGGLEHRLEAACNWTYGERCDLEDRDRAELSPLLHTTTQWAILPCSFTPTPALSTGLIHLHQNIVDTQYLYGLSSSIVSWAVKWEYIMLVFLLLADARICTCLLILLLICQAEA

SEQ ID NO: 13 - protein, 391 aa, Hepatitis C virus
  MISC_FEATURE (1)..(391): Q5I2N3[384-751], Genome polyprotein, Hepatitis C virus genotype 6a (isolate 6a33)
YFGMAGNWLKVLAVLFLFAGVEATTTVGHGVARTTAGITGLFSPGASQNLQLIKNGSSWHINRTALNCNDSLQTGFLASLFYVRKFNSSGCPERMAVCKSLADFRQGWGQITYKVNISGPSDDRPYCWHYAPRPCDVVPASTVCGPVYCFTPSPVVIGTTDRRGNPTYTWGENETDVFMLESLRPPTGGWFGCTWMNSTGFTKTCGAPPCQIIPGDYNSSANELLCPTDCFRKHPEATYQRCGSGPWVTPRCLVDYPYRLWHYPCTVNFTVHKVRMFVGGIEHRFDAACNWTRGERCELHDRDRIEMSPLLFSTTQLAILPCSFSTMPALSTGLIHLHQNIVDVQYLYGVSSSVTSWVVKWEYIVLMFLVLADARICTCLWLMLLISNVEA

SEQ ID NO: 14 - protein, 368 aa, Hepatitis C virus
  MISC_FEATURE: O92529[384-751], Genome polyprotein, Hepatitis C virus genotype 6b (isolate Th580)
TTTVGRAAGRSAYLFTSIFSSGPNQKIQLINTNGSWHINRTALNCIDSLQTGFLSALFYRSNFNSTGCSERLGACKPLEHFQQGWGPITHKSNITGPSEDRPYCWHYAPRECSVVPASSVCGPVYCFTPSPVVVGTTDRLGNPTYNWGENETDVFMLESLRPPQGGWFGCTWMNSTGFTKTCGAPPCQLIPGDYNSSSNQLLCPTDCFRKHPEATYQKCGSGPWLTPRCLVDYPYRLWHYPCTVNYTIHKVRMFIGGVEHRFDAACNWTRGDRCDLYDRDRIEMSPLLFSTTQLAILPCSFTTMPALSTGLIHLHQNIVDVQYLYGVSSSIVSWAVKWEYVVLMFLVLADARICTCLWLMLLVGKVEA

(19) United States
(12) Patent Application Publication
(10) Pub. No.: US 2016/0361311 A1
(43) Pub. Date: Dec. 15, 2016

(54) LIGANDS THAT TARGET HEPATITIS C VIRUS E2 PROTEIN

(71) Applicants: Rodney BALHORN, Livermore, CA (US); AMERICAN UNIVERSITY OF CAIRO, New Cairo (EG)

(72) Inventors: Reem AL-OLABY, Cairo (EG); Hassan AZZAZY, Alexandria (EG); Rodney BALHORN, Livermore, CA (US)

(73) Assignee: AMERICAN UNIVERSITY OF CAIRO, New Cairo (EG)

(21) Appl. No.: 15/121,691

(22) PCT Filed: Feb. 25, 2015
(86) PCT No.: PCT/IB2015/000979

§ 371 Date: Aug. 25, 2016

Related U.S. Application Data
(60) Provisional application No. 61/944,422, filed on Feb. 25, 2014.

(51) Int. Cl. A61K 31/501; A61K 31/47; A61K 31/404; A61K 31/519; A61K 31/422; A61K 31/566; A61K 31/407; A61K 31/4409; A61K 31/255; A61K 31/167; A61K 31/42; A61K 31/403; A61K 31/517; A61K 31/4745; A61K 31/513; A61K 31/366; A61K 31/122; A61K 47/48; A61K 31/53

(52) U.S. Cl. CPC A61K 31/501; A61K 31/53; A61K 31/47; A61K 31/404; A61K 31/519; A61K 31/422; A61K 31/566; A61K 31/407; A61K 31/4409; A61K 31/255; A61K 31/167; A61K 31/42; A61K 31/403; A61K 31/517; A61K 31/4745; A61K 31/513; A61K 31/366; A61K 31/122; A61K 47/481

(57) ABSTRACT

Hepatitis C Virus (HCV) infects 200 million individuals worldwide. Although several FDA approved drugs targeting the HCV serine protease and polymerase have shown promising results, there is a need for better drugs that are effective in treating a broader range of HCV genotypes and subtypes without being used in combination with interferon and/or ribavirin. Recently, the crystal structure of the core of the HCV E2 protein (E2c) has been determined, providing structural information that can now be used to target the E2 protein and develop drugs that disrupt the early stages of HCV infection by blocking E2's interaction with different host factors. By targeting sites containing conserved E2 amino acids in the CD81 binding site on HCV E2, one might also be able to develop drugs that block HCV infection in a genotype-independent manner. Using the E2c structure as a template, a structural model of the E2 protein core (residues 421-645) was developed that includes the three amino acid segments that are not present in the E2c crystal structure. Blind docking of a diverse library of 1715 small molecules to this model led to the identification of a set of 34 ligands predicted to bind near conserved amino acid residues involved in the HCV E2:CD81 interaction. Surface plasmon resonance was used to screen the ligand set for binding to recombinant E2 protein, and the best binders were subsequently tested to identify compounds that inhibit the infection of hepatocytes by HCV. One compound, 281816, blocked E2 binding to CD81 and inhibited hepatocyte infection by HCV genotypes 1a, 1b, 2a, 2b, 4a and 6a with IC50's ranging from 2.2 μM to 4.6 μM. Methods are described for preventing or treating HCV infection using small molecule inhibitors such as 281816 that target E2 and disrupt its interactions.

### CROSS-REFERENCE TO RELATED APPLICATION

This application claims priority benefit to U.S. Provisional Application No. 61/944,422, filed Feb. 25, 2014 which is incorporated by reference in its entirety.

### BACKGROUND OF THE INVENTION

Field of the Invention

A new Hepatitis C Virus E2 Protein (“HCV E2 Protein”) model was created based on the E2c crystal structure. Blind docking to this model identified small molecules that bind to HCV E2 protein, that block attachment of HCV E2 protein to CD81, a ligand expressed on cells infected by HCV, and that block infection of hepatocytes by HCV. Such ligands were shown to block attachment to and infection of host cells by more than one genotype of HCV. The invention pertains to small molecule ligands, homo- or hetero-multimeric ligands, and ligand conjugates that target the HCV E2 protein and to methods for inhibiting the attachment, invasion and infection of cells by HCV using these ligands to block HCV attachment to CD81 and other cellular determinants.

Description of Related Art

Hepatitis C Virus (HCV) is a global public health problem [1] in which nearly 85% of affected individuals have acute HCV infections and exhibit no symptoms. In addition, more than three-quarters of these cases will advance to chronic disease, which include liver cirrhosis and liver cancer [2]. The current standard of care treatment for HCV (Peginterferon/Ribavirin, PR) can cause deleterious side effects, and a sustained virologic response (SVR) is achieved in less than 50% of genotype-1 patients [3]. The FDA approved protease inhibitors Telaprevir (TVR) and Boceprevir (BOC) have been shown to provide higher SVR rates in genotype 1 patients [3, 4] when each is combined with PR. However the poor safety profile of TVR and BOC reported in the Week 16 analysis of the French Early Access Program suggest there is still a need for better HCV drugs [5]. The two most recent FDA approvals have been for the oral drugs Simeprevir and Sofosbuvir, inhibitors that target the HCV NS3/4A protease and polymerase, respectively [6]. Semiprevir, which needs to be administered with ribavirin and peg-interferon, has a number of undesirable side effects [7]. The efficacy of Semiprevir has also been shown to be diminished significantly, due to viral breakthrough (HCV RNA rebounds and becomes detectable in the patient before treatment is completed), in patients infected by HCV genotypes 4-6 containing the Q80K, R155K and D168E/V polymorphisms [7]. Recommendations for the use of Sofosbuvir indicate it should be administered with Ribavirin in HCV genotype 2 and 3 infections and that Peg-Interferon should be included in the treatment when infections involve genotypes 1 and 4. While Sofosbuvir is considered the Holy Grail in HCV treatment by some, it is recommended that treatments be limited to 12 weeks only [6]. Its high cost ($1,000 USD/pill) also puts it out of reach of many HCV infected patients.

Despite the advances that have been made in the field of HCV drug development, our current drugs offer little protection against the emergence of genetic variants (escape variants) of HCV—a feature of HCV biology that complicates both drug and vaccine development. Drugs that target only one step in the HCV life cycle will be the least effective in treating patients that become infected with these emerging variants. The current FDA approved drugs are good examples, as they are only effective against a subset of genotypes. This has led many of the larger pharmaceutical companies to continue developing new drugs that target one or more steps in the HCV life cycle and block virus invasion, processing of the pro-protein or replication of the viral genome.

Several research groups have reported that the CD81-large extracellular loop (CD81-LEL) plays a key role in HCV entry into cells by binding to the HCV E2 glycoprotein [15-18]. Zhang et al. [19] elucidated a separate, additional function for CD81 in the HCV life cycle. These studies showed that CD81-LEL is important for efficient HCV genome replication. In addition, the E2-CD81-LEL interaction has been shown to induce several immuno-modulatory effects such as the production and release of pro-inflammatory cytokine gamma interferon from T-cells. In addition, this interaction has also been shown to down regulate T-cell receptors and suppress the activity of natural killer (NK) cells [20]. Therefore, it is tempting to speculate that blocking CD81-LEL:HCV E2 interaction might also contribute to arresting disease progression to liver cirrhosis.

While we have known for some time that the E2 envelope glycoprotein plays an important a role in the life cycle of HCV, we are only now beginning to learn details about the structure of the protein and how it functions. This has been attributed to the challenging intrinsic properties of the HCV E2 glycoprotein, such as the presence of multiple flexible loops, its tendency to form disulfide aggregates in solution and the high level of N-linked glycosylation, all of which make it difficult to determine the protein's structure. Owsianka et al. [41] identified the amino acid residues in HCV E2 glycoprotein that interact with CD81-LEL (Q412-N423, S432-F447, L480-P493, S528-D535, P544-G551, P612-C620). Several other research groups also identified three putative CD81 binding sites on E2 that have been referred to as region 1, Y474-R492 [21, 42], region 2, S522-G551 [13,21,41-43], and region 3, P612-P619 [21,42]. Region 3 has been found to be the most conserved among these sites [43].

Several approaches are being used to develop anti-HCV drugs and vaccines that target the HCV E2 glycoprotein [8-11]. These efforts have had to deal with challenges that relate to the genomic diversity and heterogeneity of HCV, limitations in animal models used to test vaccines and drugs and the lack of a resolved crystal structure for the HCV E2 glycoprotein. Recently, Kong et al. [12] has been able to obtain information on the structure of HCV E2 (amino acid residues 384-746) by designing and expressing 41 soluble HCV E2 constructs and selecting 15 to screen against E2-specific Fab fragments in crystallization trials. Using a combination of x-ray crystallography and negative stain-electron microscopy, Kong et al. [12] discovered that the structures they obtained for E2 were globular and very different from the predicted models of E2 that were developed using class II fusion protein templates containing three β-sheet domains. In addition to providing new structural information about an important region of the E2 protein, Kong et al. also characterized the conserved neutralizing epitopes in HCV E2 by determining the crystal structure of an E2 peptide domain containing residues Q412 to N423 of HCV E2 glycoprotein bound to AP33—a broadly neutralizing antibody [13]. Additionally, they were able to identify key CD81-binding residues through mutational studies. The CD81 binding sites were determined to be in the AR3C epitope, along one side of the β-sandwich (an isolated region of the CD81-binding loop) and a front layer consisting of loops, short helices and β-sheets [12-13]. AR3C was also found to cross-neutralize HCV genotypes by blocking CD81 binding to HCV E2 [14].

The determination and recent publication of this HCV E2 protein core structure [12] has made it possible to use computational docking and structure-based drug design methods to begin developing anti-HCV drugs that target the conserved regions of the HCV E2 glycoprotein and block E2's interaction with host receptors. These methods could not, however, be applied directly to the new E2c crystal structure because the structure is missing three important peptide segments P453-P491, V574-N577, and P586-R596 and a number of the amino acids in its sequence are not found in the HCV genotype 1a protein. Nevertheless, the inventors were able to create a high quality homology model of the E2 protein core (FIG. 1) suitable for small molecule docking that contains these missing segments and the correct genotype 1a amino acid sequence. This model was used to identify small molecule drug leads that target highly conserved sites on the HCV E2 glycoprotein located within the region bound by CD81. AutoDock was used to perform virtual screening runs against 1715 small molecules and 34 of the best compounds were tested experimentally using surface plasmon resonance (Biacore T100) to identify a set of small molecules that bind to recombinant E2 protein. The compounds showing binding activity were then tested for their ability to block HCV infection of hepatocytes. One compound, 281816, was found to block infection of hepatocytes by each of the HCV genotypes and subtypes tested (1a, 1 b, 2a, 2b, 4a and 6a) in a dose-dependent manner.

### BRIEF SUMMARY OF THE INVENTION

Using the E2c structure as a template, the inventors developed a structural model of the E2 protein core (residues 421-645) that includes the three amino acid segments that are not present in the E2c crystal structure. Blind docking of a diverse library of 1715 small molecules to this model led to the identification of a set of 34 ligands predicted to bind near conserved amino acid residues involved in the HCV E2: CD81 interaction. Surface plasmon resonance was used to screen the ligand set for binding to recombinant E2 protein, and the best binders were subsequently tested to identify compounds that inhibit the infection of hepatocytes by HCV. One compound, 281816, blocked E2 binding to CD81 and inhibited hepatocyte infection by HCV genotypes 1a, 1b, 2a, 2b, 4a and 6a with IC50's ranging from 2.2 μM to 4.6 μM. These results suggest the development of small molecule inhibitors such as 281816 that target E2 and disrupt its interactions may provide a new paradigm for HCV treatment.

Five sites on the surface of the E2 protein model were used to select a set of small molecules predicted by blind docking to bind to locations that could interfere with E2 binding to CD81 (FIGS. 2-6). Cavities on the E2 protein were selected to contain or to be located adjacent to amino acids previously identified to participate in the binding of E2 to CD81 based on the epitopes of neutralizing antibody and mutagenesis studies [12, 31-35]. Following the identification of a set of 34 ligands predicted to bind to these sites, the compounds were tested experimentally using surface plasmon resonance to identify ligands that bind to recombinant E2 immobilized on a chip. Twenty-three of the compounds (67% of those tested) were observed to bind to the recombinant E2 protein. All 23 ligands, described in Table 1, were predicted to bind to one or more of the five sites.

The majority were predicted to bind to several sites of interest. As is typical for docking studies with small ligands, predictions of ligand binding identified multiple ligand conformations predicted to bind within each binding site. Ligand 281816, for example, was predicted to bind to two different locations on E2 shown in FIG. 9. One cluster of molecules bound deep inside a cavity positioned directly above Y618 and P619, two amino acids in site 4 that are known to contribute to E2's binding to CD81. A subset of the molecules in this cluster bound to site 2 and immediately adjacent to D481-P490, part of the epitope targeted by antibodies that block HCV infectivity but have no effect on E2 binding to CD81. W487, whose mutation has shown the amino acid residue to participate in E2:E1 dimerization, was also located near this site 2 cluster. 281816 molecules in the second cluster bound to a more shallow cavity on the opposite of the protein. These molecules are located in site 1, directly above residues R614-W616 and immediately adjacent to P612, Y613 and H421-N423. These amino acid residues have been shown to be critical for E2 binding to CD81. As expected, the ligand positioned above Y618 and P619 in the deeper cavity was predicted to bind more strongly to the protein (free energy of the best bound ligand=−8.64 Kcal/mol Å³) than when it was bound to the more shallow cavity on the other side of the protein (free energy=−6.39 Kcal/mol Å³). One interesting and unique feature of the 281816 ligand is that it is predicted to bind immediately above or adjacent to both of the exposed faces of the P612-P619 domain that is known to participate in E2 binding to CD81.

When the 23 ligands were tested for activity in blocking HCV infection of hepatocytes, ligand 281816 was found to inhibit HCV infection using both HCVcc (Hepatatis C Virus-cell culture derived) and HCVpp (Hepatitis C Virus pseudoparticle) based assays. Upon analyzing the activity spectrum of HCV using HCVpp bearing envelope proteins from different HCV genotypes (1a, 1b, 2a, 2b, 4a and 6a), 281816 was found to inhibit the infection of all tested genotypes with IC50's ranging from 2.2 μM to 4.6 μM (Table 4), indicating that this small molecule inhibits HCV entry in a genotype-independent manner. Flow cytometry experiments conducted with 281816 also confirmed the ligand was effective in blocking the binding of E2 protein to native CD81 on cells in culture. These observations are consistent with the results obtained in silico which predicted the binding of 281816 to sites on E2 involving conserved residues in the CD81 binding site of HCV E2 that have been shown by mutagenesis studies to be essential for HCV infection. The results also show that docking experiments conducted with the new homology model for E2 (a model that contains the peptide segments missing in the recent E2c crystal structure) developed by the inventors can be used to identify small molecules that target and bind to important sites on the HCV E2 glycoprotein. Both the docking and experimental studies identified 281816 as a promising new drug lead for treating HCV through the inhibition of an early step in HCV infection, the binding of the viral E2 protein to the CD81 receptor on hepatocytes.

Specific, nonlimited embodiments of the invention include:
1. A small organic molecule that binds to Hepatitis C Virus (HCV) E2 protein or a multimer or conjugate comprising said small organic molecule.
2. The small organic molecule of embodiment 1 that binds to at least one of Sites 1, 2, 3, 4 or 5.
3. The small organic molecule of embodiment 1 that comprises core structure 1, 2, 3 or 4.
4. The small organic molecule of embodiment 1 selected from the group consisting of 3076, 4429, 57103, 60785, 81462, 84100, 86467, 113486, 117268, 121861, 133071, 144694, 146554, 158413, 171303, 204232, 211490, 213700, 215629, 281254, 281816, 308835, 359472, 403379, 639174, and 6702831; or a molecule that binds at a site surrounded by or adjacent to at least four, five, six, seven or more of the same amino acid residues on HCV E2 protein as said molecule.
5. The molecule of embodiment 1 selected from the group consisting of 281816, 73735, 57103, and 133071; or a molecule that binds at a site surrounded by or adjacent to at least four, five, six, seven or more of the same amino acid residues on HCV E2 protein as said molecule.
6. The molecule of embodiment 1 that is 281816; or a molecule that binds at a site surrounded by or adjacent to at least four, five, six, seven or more of the same amino acid residues on HCV E2 protein as said molecule.
7. A multimer comprising at least two, three, four, five, six, seven, eight, nine, or ten or more molecules of embodiment 1 linked together, optionally via a linker or carrier.
8. A multimer comprising at least two, three, four, five, six, seven, eight, nine, or ten or more molecules of embodiment 2 linked together, optionally via a linker or carrier.
9. A multimer comprising at least two, three, four, five, six, seven, eight, nine, or ten or more molecules of embodiment 3 linked together, optionally via a linker or carrier.
10. A multimer comprising at least two, three, four, five, six, seven, eight, nine, or ten or more molecules of embodiment 4 linked together, optionally via a linker or carrier.
11. A multimer comprising at least two, three, four, five, six, seven, eight, nine, or ten or more molecules of embodiment 5 linked together, optionally via a linker or carrier.
12. A multimer comprising at least two, three, four, five, six, seven, eight, nine, or ten or more molecules of embodiment 6 linked together, optionally via a linker or carrier.
13. A conjugate comprising at least one, two, three, four, five, six, seven, eight, nine, ten or more molecules of embodiment 1 linked to at least one, two, three, four, five, six, seven, eight, nine, ten or more other molecules that bind to HCV E2 protein, optionally via linker(s) or carrier(s).
14. A conjugate comprising at least one, two, three, four, five, six, seven, eight, nine, ten or more molecules of embodiment 2 linked to at least one, two, three, four, five, six, seven, eight, nine, ten or more other molecules that bind to HCV E2 protein, optionally via linker(s) or carrier(s).
15. A conjugate comprising at least one, two, three, four, five, six, seven, eight, nine, ten or more molecules of embodiment 3 linked to at least one, two, three, four, five, six, seven, eight, nine, ten or more other molecules that bind to HCV E2 protein, optionally via linker(s) or carrier(s).
16. A conjugate comprising at least one, two, three, four, five, six, seven, eight, nine, ten or more molecules of embodiment 4 linked to at least one, two, three, four, five, six, seven, eight, nine, ten or more other molecules that bind to HCV E2 protein, optionally via linker(s) or carrier(s).
17. A conjugate comprising at least one, two, three, four, five, six, seven, eight, nine, ten or more molecules of embodiment 5 linked to at least one, two, three, four, five, six, seven, eight, nine, ten or more other molecules that bind to HCV E2 protein, optionally via linker(s) or carrier(s).
18. A conjugate comprising at least one, two, three, four, five, six, seven, eight, nine, ten or more molecules of embodiment 6 linked to at least one, two, three, four, five, six, seven, eight, nine, ten or more other molecules that bind to HCV E2 protein, optionally via linker(s) or carrier(s).
19. A conjugate comprising at least one, two, three, four, five, six, seven, eight, nine, ten or more molecule(s) according to embodiment 1 linked to at least one, two, three, four, five, six, seven, eight, nine, ten or more molecule(s) that binds to CD81, optionally via a linker(s) or carrier(s).
20. A conjugate comprising at least one, two, three, four, five, six, seven, eight, nine, ten or more molecule(s) according to embodiment 2 linked to at least one, two, three, four, five, six, seven, eight, nine, ten or more molecule(s) that binds to CD81, optionally via a linker(s) or carrier(s).
21. A conjugate comprising at least one, two, three, four, five, six, seven, eight, nine, ten or more molecule(s) according to embodiment 3 linked to at least one, two, three, four, five, six, seven, eight, nine, ten or more molecule(s) that binds to CD81, optionally via a linker(s) or carrier(s).
22. A conjugate comprising at least one, two, three, four, five, six, seven, eight, nine, ten or more molecule(s) according to embodiment 4 linked to at least one, two, three, four, five, six, seven, eight, nine, ten or more molecule(s) that binds to CD81, optionally via a linker(s) or carrier(s).
23. A conjugate comprising at least one, two, three, four, five, six, seven, eight, nine, ten or more molecule(s) according to embodiment 5 linked to at least one, two, three, four, five, six, seven, eight, nine, ten or more molecule(s) that binds to CD81, optionally via a linker(s) or carrier(s).
24. A conjugate comprising at least one, two, three, four, five, six, seven, eight, nine, ten or more molecule(s) according to embodiment 6 linked to at least one, two, three, four, five, six, seven, eight, nine, ten or more molecule(s) that binds to CD81, optionally via a linker(s) or carrier(s).
25. The use of at least one molecule according to embodiment 1 for the preparation of a medicament for preventing or treating HCV infection.
26. The use of at least one molecule according to embodiment 2 for the preparation of a medicament for preventing or treating HCV infection.
27. The use of at least one molecule according to embodiment 3 for the preparation of a medicament for preventing or treating HCV infection.
28. The use of at least one molecule according to embodiment 4 for the preparation of a medicament for preventing or treating HCV infection.
29. The use of at least one molecule according to embodiment 5 for the preparation of a medicament for preventing or treating HCV infection.
30. The use of at least one molecule according to embodiment 6 for the preparation of a medicament for preventing or treating HCV infection.
31. A composition comprising at least two molecules according to embodiment 1 and a pharmaceutically acceptable carrier or excipient.
32. A composition comprising at least two molecules according to embodiment 2 and a pharmaceutically acceptable carrier or excipient.
33. A composition comprising at least two molecules according to embodiment 3 and a pharmaceutically acceptable carrier or excipient.
34. A composition comprising at least two molecules according to embodiment 4 and a pharmaceutically acceptable carrier or excipient.
35. A composition comprising at least two molecules according to embodiment 5 and a pharmaceutically acceptable carrier or excipient.
36. A composition comprising at least two molecules according to embodiment 6 and a pharmaceutically acceptable carrier or excipient.
37. A method for preventing a subject at risk of HCV infection from being infected with HCV comprising administering an amount of at least one compound of embodiment 1, or a multimer or conjugate thereof, to a subject in need thereof in an amount effective to prevent HCV infection.
38. A method for preventing a subject at risk of HCV infection from being infected with HCV comprising administering an amount of at least one compound of embodiment 2, or a multimer or conjugate thereof, to a subject in need thereof in an amount effective to prevent HCV infection.
39. A method for preventing a subject at risk of HCV infection from being infected with HCV comprising administering an amount of at least one compound of embodiment 3, or a multimer or conjugate thereof, to a subject in need thereof in an amount effective to prevent HCV infection.
40. A method for preventing a subject at risk of HCV infection from being infected with HCV comprising administering an amount of at least one compound of embodiment 4, or a multimer or conjugate thereof, to a subject in need thereof in an amount effective to prevent HCV infection.
41. A method for preventing a subject at risk of HCV infection from being infected with HCV comprising administering an amount of at least one compound of embodiment 5, or a multimer or conjugate thereof, to a subject in need thereof in an amount effective to prevent HCV infection.
42. A method for preventing a subject at risk of HCV infection from being infected with HCV comprising administering an amount of at least one compound of embodiment 6, or a multimer or conjugate thereof, to a subject in need thereof in an amount effective to prevent HCV infection.
43. A method for treating a subject infected with HCV comprising administering an amount of at least one compound of embodiment 1, or a multimer or conjugate thereof, to a subject in need thereof in an amount effective to reduce the severity of HCV infection.
44. A method for treating a subject infected with HCV comprising administering an amount of at least one compound of embodiment 2, or a multimer or conjugate thereof, to a subject in need thereof in an amount effective to reduce the severity of HCV infection.
45. A method for treating a subject infected with HCV comprising administering an amount of at least one compound of embodiment 3, or a multimer or conjugate thereof, to a subject in need thereof in an amount effective to reduce the severity of HCV infection.
46. A method for treating a subject infected with HCV comprising administering an amount of at least one compound of embodiment 4, or a multimer or conjugate thereof, to a subject in need thereof in an amount effective to reduce the severity of HCV infection.
47. A method for treating a subject infected with HCV comprising administering an amount of at least one compound of embodiment 5, or a multimer or conjugate thereof, to a subject in need thereof in an amount effective to reduce the severity of HCV infection.
48. A method for treating a subject infected with HCV comprising administering an amount of at least one compound of embodiment 6, or a multimer or conjugate thereof, to a subject in need thereof in an amount effective to reduce the severity of HCV infection.
49. A method for inhibiting or blocking the attachment of HCV to a cell comprising contacting HCV with at least one compound of embodiment 1, or a multimer or conjugate thereof
50. A method for inhibiting or blocking the attachment of HCV to a cell comprising contacting HCV with at least one compound of embodiment 2, or a multimer or conjugate thereof
51. A method for inhibiting or blocking the attachment of HCV to a cell comprising contacting HCV with at least one compound of embodiment 3, or a multimer or conjugate thereof.
52. A method for inhibiting or blocking the attachment of HCV to a cell comprising contacting HCV with at least one compound of embodiment 4, or a multimer or conjugate thereof.
53. A method for inhibiting or blocking the attachment of HCV to a cell comprising contacting HCV with at least one compound of embodiment 5, or a multimer or conjugate thereof
54. A method for inhibiting or blocking the attachment of HCV to a cell comprising contacting HCV with at least one compound of embodiment 6, or a multimer or conjugate thereof.

### BRIEF DESCRIPTION OF FIGURES

FIG. 1. Structural model of HCV E2. This model was developed using the E2c crystal structure reported by Kong et al. [12] as the template. The sequence modeled contains amino acid residues H421 through N645. The core of the protein structure provided by the E2c structure is shown in white. The peptide segments that are not present in the E2c crystal structure are shown in red (P453-P491), blue (V574-N577) and green (F586-R596).

FIG. 2. Site 1 of the E2 Protein. The amino acids surrounding this site are colored black and labeled. This site is sandwiched between H421-N423, a set of amino acids that have been shown by mutation studies to be important for infectivity and P612, Y613, and H617-P619, a group of amino acids known to be critical for E2 binding to CD81.

FIG. 3. Site 2 of the E2 Protein. The amino acids surrounding this site are colored black and labeled. This site is positioned immediately adjacent to amino acids Y474-R492, a region of the protein has no effect on E2 binding CD81 but antibodies binding to this peptide segment prevent infectivity. One amino acid within this segment, W487, is a key amino acid that is involved in E2 binding to E1.

FIG. 4. Site 3 of the E2 Protein. The amino acids surrounding this site are colored black and labeled. The majority of the amino acids (S522-G551) located in or around this site have been shown to be critical for E2 binding to CD81.

FIG. 5. Site 4 of the E2 Protein. The amino acids surrounding this site are colored black and labeled. Amino acids within this region, which include P612, Y613, and H617-P619), are known to be critical for E2 binding to CD81.

FIG. 6. Site 5 of the E2 Protein. The amino acids surrounding this site are colored black and labeled. Amino acids P612 and Y613 next to this site are critical for E2 binding to CD81.

FIG. 7. Comparison of the crystal structure of E2c with the homology model. A. E2c crystal structure (red/gray). B. Homology model (black). C. Superposition of the two structures. The residues colored red remain unchanged from the crystal structure. The residues colored black show the structure is either different or not present in the crystal structure.

FIG. 8. Surface Plasmon resonance sensogram (Biacore T100). This figure shows sensorgrams (binding and dissociation plots) for two of the ligands that bound to the recombinant E2 protein immobilized on a CM5 chip, 86467 (green/lower trace) and 121861 (red/upper trace), and the 3 reference points that are used to measure the binding and dissociation (dissociation 1 and dissociation 2) of the compound expressed in response units (RU).

FIG. 9. 281816 binding sites on HCV E2. 281816 (structure, top) is predicted to bind to two sites on the E2 protein. Clusters of different conformations of bound 281816 molecules are shown bound in the two sites. Color coding for amino acids and peptide segments shown to be important for HCV infectivity or binding to CD81 are: H421-N423 (yellow), Y474-R492 (light cyan), W487 (dark cyan), S522-G551 (light green), Y527 and W529 (dark green), P612, Y613, and H617-P619 (red), and R614-W616 (pink).

FIG. 10. 281816 inhibits HCV entry in a genotype-independent manner. (A) Huh-7 cells in 96-well plates were pre-treated with 281816 compound (left and middle panels) or anti-CD81 antibody (right panel) at the indicated concentrations and then infected with HCVpp 2a or HCVcc. (B) Huh-7 cells in 24-well plates were pre-treated with 281816 compound at the indicated concentrations and infected with HCVpp expressing envelope proteins from the indicated genotype. After 30 hours of infection, cells were lysed and luciferase activity quantified. HCVpp infections were normalized to RD114pp infections.

FIG. 11. 281816 inhibition of HCV E2 protein binding to CD81 on Raji cells. Flow cytometry was used to quantify recombinant HCV E2 protein binding to native CD81 over-expressed on Raji cells. Binding of the recombinant E2 protein to native CD81 on the surface of Raji cells was detected using the mouse monoclonal E2 antibody clone H53 followed by staining with a secondary anti mouse-FITC antibody as described in the Materials and Methods section. E2 binding is inhibited by 281816 in a dose-dependent manner up to 100 μM.

FIG. 12. Spatial depiction of adjacent ligands for HCV E2 that could be chemically linked to each other to provide a conjugate having higher affinity for HCV E2.

FIG. 13. Example of an HCV E2 ligand dimer showing the structure of one possible dimer of ligand 281816.

FIG. 14. Example of an HCV E2 ligand trimer showing the structure of one possible trimer of ligand 281816.

FIG. 15. Example of an HCV E2 ligand conjugate containing two different HCV E2 ligands. The conjugate shown is produce by linking together ligand 281816 and ligand 146554.

### DETAILED DESCRIPTION OF THE INVENTION

One of the best approaches for developing therapeutics that prevent HCV infection is to block the initial infection of the host cells by the virus. For HCV, this can be accomplished by inhibiting the interaction between the HCV E2 glycoprotein located on the surface of the virus and CD81, a cell surface receptor found on hepatocytes, since the binding of the E2 protein to CD81 plays a key role in viral infection. Sites on CD81 that are known to interact with E2 were targeted and the inventors previously identified a series of small molecules/ligands that bound to these regions and could be used (without modification or as parent compounds for use in fragment based extension or linking ligands together) to create more effective inhibitors. Here we use a different structural model, but a similar functional approach to identify small molecules/ligands that bind to the HCV E2 protein in the region that interacts with CD81. This has not been accomplished previously because of lack of useful model and sufficient structural information from crystal or NMR structures for discovery or design of compounds that bind to the E2 protein.

In order to use computational docking methods to identify drug candidates that bind to the HCV E2 protein and block the E2-CD81 interaction, the inventors developed a new HCV E2 homology model using the AS2TS modeling method. This model permitted the identification of a series of small molecules that not only bind to HCV E2 protein, but a subset of these molecules that also inhibit the infection of hepatocytes by HCV. The structural template was the envelope glycoprotein of the tick-borne encephalitis virus (PDB entry: 1SVB). We then identified the amino acid residues in the model that interact with CD81-LEL and neutralizing antibodies based on previous literature, defined three grid boxes surrounding these regions on the protein (model) surface using Autodock, docked a set of 3,000 ligands to the protein within the gridded regions and identified top virtual screening hits, tested the ligands experimentally using surface plasmon resonance (Biacore t100) to determine which ones bind to the recombinant HCV E2 protein, and then tested the ligands using HCVcc and HCVpp assays to determine which ones block HCV virus invasion and infection. Out of the seven ligands that were tested, four ligands were found to exert an inhibitory effect on HCV infectivity with an IC50 ranging from 0.3 uM to 6 μM. The table below describes ligands to HCV E2 protein.

| E2 Ligand | | |
|---|---|---|
| NCI # | Chemical Name | Structure |
| 3076 | (4R)-6-N,4-diphenyl-1,4- dihydro-1,3,5-triazine- 2,6-diamine | 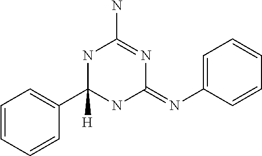 [SMILES: NC1=N/C(=N\c2ccccc2)N[C@H](c2ccccc2)N1] |
| 4429 | 6-[2-(4,6-diamino-1,3,5- triazin-2-yl)phenyl]-1,3,5- triazine-2,4-diamine | 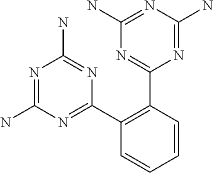 [SMILES: Nc1nc(N)nc(-c2ccccc2-c2nc(N)nc(N)n2)n1] |
| 57103 | 5,7-dinitroquinolin-8- olate | 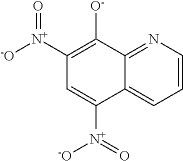 |
| 60785 | ZINC13154304 | 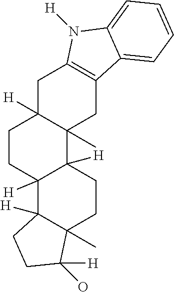 [SMILES: CC12CCC3C(CCC4Cc5[nH]c6ccccc6c5CC43C)C1CCC2O] |
| 81462 | ZINC19325788 | 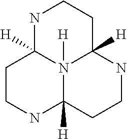 |
| 84100 | (2S)-5-phenyl-2-[(2S)-5- phenyl-2,3-dihydro-1,3- benzoxazol-2-yl]-2,3- dihydro-1,3-benzoxazole | 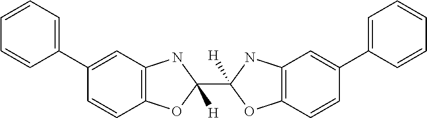 [SMILES: c1ccc(-c2ccc3c(c2)N[C@H]([C@H]2Nc4cc(-c5ccccc5)ccc4O2)O3)cc1] |
| 86467 | 2-[[(8R,9S,13S,14S)-13- methyl-17-oxo- 7,8,9,11,12,14,15,16- octahydro-6H- cyclopenta[a]phenanthren- 3-yl]oxy]acetate | 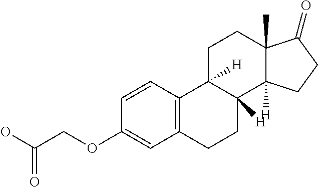 |
| 113486 | 2-(5,6-dimethyl-4-oxo- 1H-pyrimidin-2-yl)-1,1- dimethylguanidine | 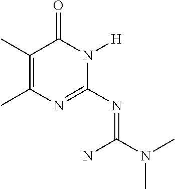 |
| 117268 | 4-[(4R)-2,6-diamino-4-(4- chlorophenyl)-1,4- dihydro-1,3,5-triazin-3- ium-3- yl]benzenesulfonamide | 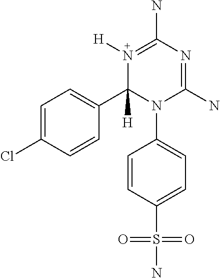 |
| 121861 | ZINC01711028 | 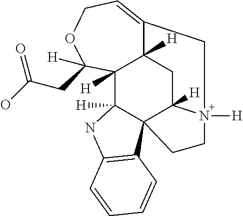 |
| 133071 | 3-[3-chloro-4-(3- phenoxypropoxy)phenyl]- 4,4-dimethyl-1H-1,3,5- triazin-3-ium-2,6-diamine | 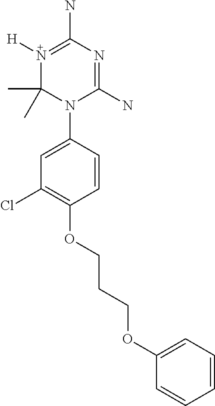 |
| 144694 | (E)-2-naphthalen-1-yl-3- pyridin-4-ylprop-2- enamide | 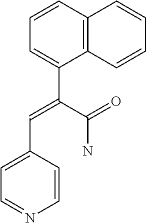 [SMILES: NC(=O)/C(=C\c1ccncc1)c1cccc2ccccc12] |
| 146554 | 1-(2-methyl-5- nitrophenyl)-3-(4- sulfamoylphenyl)urea | 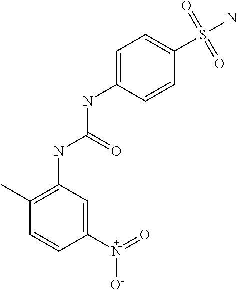 |
| 158413 | 4-chloro-2-(3- phenylsulfanylphenyl) quinoline-6- sulfonyl fluoride | 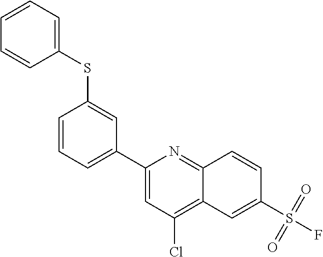 [SMILES: O=S(=O)(F)c1ccc2nc(-c3cccc(Sc4ccccc4)c3)cc(Cl)c2c1] |
| 171303 | 3-nitro-N-(5-nitro-1,3- thiazol-2-yl)benzamide | 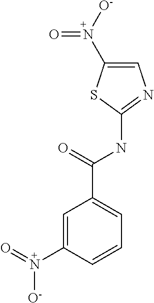 |
| 204232 | 1-N,3-N-bis(3- nitrophenyl)benzene-1,3- dicarboxamide | 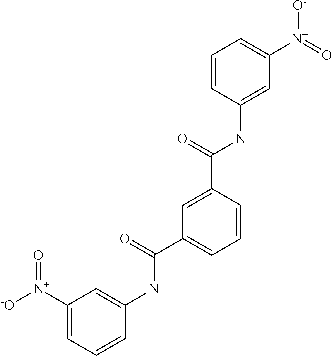 |
| 211490 | (4S)-2,4-bis(3,4- dimethoxyphenyl)-4H- pyrano[3,2-c]chromen-5- one | 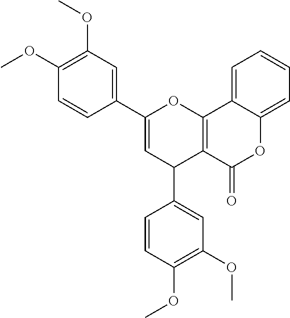 [SMILES: COc1ccc(C2=CC(c3ccc(OC)c(OC)c3)c3c(c4ccccc4oc3=O)O2)cc1OC] |
| 213700 | 1-(3,4-dimethyl-1,2- oxazol-5-yl)-3-(2-methyl- 5-nitrophenyl)urea | 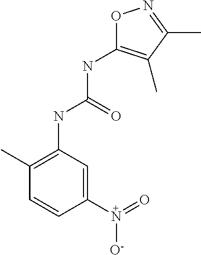 |
| 215629 | N-(4-fluoro-3- nitrophenyl)-1,3-dioxo-2- benzofuran-5- carboxamide | 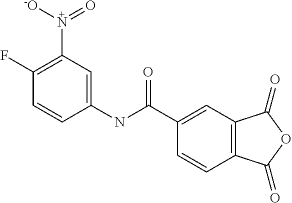 |
| 281254 | (2-oxo-1H-pyridin-3-yl) 3,5-dinitrobenzoate | 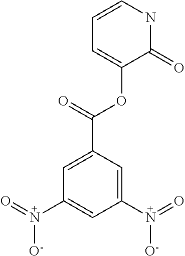 |
| 281816 | 1-methyl-4-[(5S)-3- methylsulfanyl-5,6- dihydrobenzo[b][1] benzothiepin-5-yl] piperazin-1-ium | 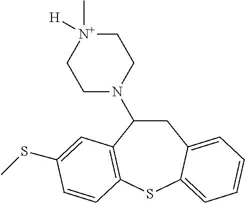 [SMILES: CSc1ccc2c(c1)C(N1CC[NH+](C)CC1)Cc1ccccc1S2] |
| 308835 | 2-[4-[4-(1,3-dioxo-2- azaspiro[4.4]nonan-2-yl)- 3-methylphenyl]-2- methylphenyl]-2- azaspiro[4.4]nonane-1,3- dione | 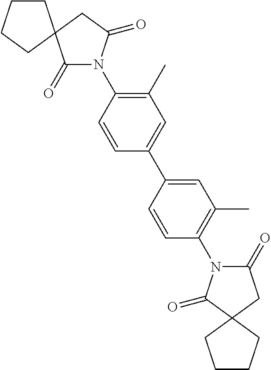 [SMILES: Cc1cc(-c2ccc(N3C(=O)CC4(CCCC4)C3=O)c(C)c2)ccc1N1C(=O)CC2(CCCC2)C1=O] |
| 359472 | N-(4-benzamido-6- propyl-1,3,5-triazin-2- yl)benzamide | 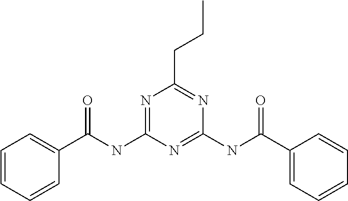 [SMILES: CCCc1nc(NC(=O)c2ccccc2)nc(NC(=O)c2ccccc2)n1] |
| 403379 | 2-amino-3- (diaminomethylidene) quinazolin-3-ium-4-one | 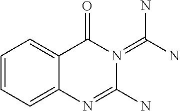 |
| 639174 | ZINC13000556 | 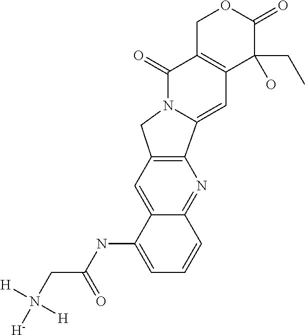 |
| 670283 | 2,2′-spirobi[3,6,7,8- tetrahydro-1H- cyclopenta[g]naphthalene]- 5,5′-dione | 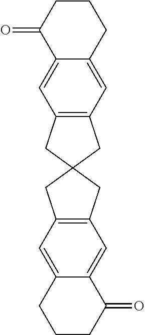 [SMILES: O=C1CCCc2cc3c(cc21)CC1(C3)Cc2cc3c(cc2C1)C(=O)CCC3] |

Many of these ligands share core structures. Representative core structures 1, 2, 3 and 4 are described below. Compounds with the different atoms or functional groups listed for X, Y, Z in these core structures are expected, based on our modeling and docking experiments, to bind as well, or better, to the E2 protein by contributing additional interactions with amino acids residues on the protein's surface. The R-R6 functional groups listed are included as modifications that are expected to have a number of effects, which include a) enhancing the compound's binding to the E2 protein (through the formation of additional hydrogen bonds, electrostatic or VanderWaals interactions), which should lowering the IC50 of the drug and reduce the required treatment dose, b) conferring or improving specific properties such as solubility or stability, c) affect the pharmacokinetics, biodistribution, absorption, tissue uptake, residence time in tissue, d) minimize toxicity, excretion or metabolism, e) enable the small molecule ligand to be conjugated to other molecules, or f) facilitate the diagnostic use of the small molecule ligand.

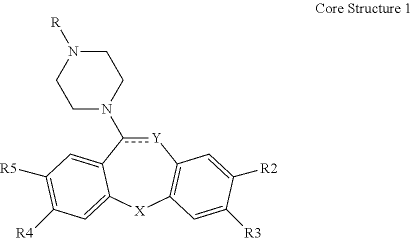

wherein:

### X=C, S, N, O, —S═O, CH2SO2H, or CH2SO2CH3;

### Y=C, S, N, or O;

R=—H, —CH2CH2OH, —CH2CH2OCH2CH2OH, —CH═O, —(CH2)xNH2, —(CH2)xCO2H, —((CH2)2O)xNH2, —((CH2)2O)xCO2H, —CH3, or —CH2CH3; where x=1, 2, 3, 4, 5, 6, 7, or 8.
R2=H, Cl, F, Br, I, —HC═O, —CO2H, —OH, ═O, —NH2, —RC═O, —CH(CH3)2, —CH3S═O, —CH3SO2, —CH═O, —CF3, —CH3, —CBr3, —CI3, CCl3, —SO2H, —SO2CH3, —SCH3, or —CH2CH3;
R3=H, Cl, F, Br, I, —HC═O, —CO2H, —OH, ═O, —NH2, —RC═O, —CH(CH3)2, —CH3S═O, —CH3SO2, —CH═O, —CF3, —CH3, —CBr3, —CI3, CCl3, —SO2H, —SO2CH3, —SCH3, or —CH2CH3;
R4=H, Cl, F, Br, I, —HC═O, —CO2H, —OH, ═O, —NH2, —RC═O, —CH(CH3)2, —CH3S═O, —CH3SO2, —CH═O, —CF3, —CH3, —CBr3, —CI3, CCl3, —SO2H, —SO2CH3, —SCH3, or —CH2CH3

### R5=H, Cl, F, Br, I, —HC═O, —CO2H, —OH, ═O, —NH2, —RC═O, —CH(CH3)2, —CH3S═O, or —CH3SO2.

Examples of Compounds Having Core Structure 1:

ZINC codes: 19362650 (corresponding to ligand 281816 that blocks HCV virus infection), 0000931, 0001639, 19632628, 19802239, 19802414, 19802374, 31554426, 19369143, 22859648, 33754515, 19801738, 33868652, 19368911, 22459325, 26185346, and 26247473.

Some representative structures for compounds having Core Structure 1 are shown below.

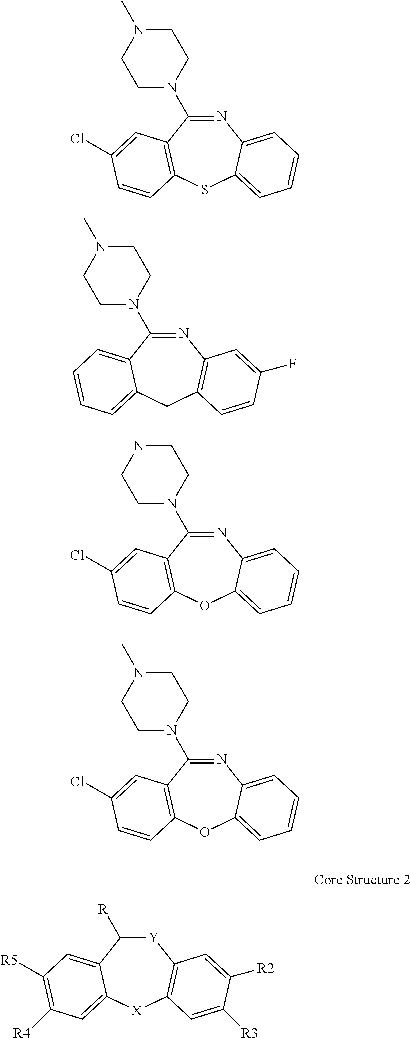

wherein

### X=C, S, N, O, S═O, CSO2H, or CSO2CH3;

### Y=C, S, N, or O;

R=—H, —CH2CH2OH, —CH2CH2OCH2CH2OH, —CH═O, —(CH2)xNH2, —(CH2)xCO2H, —((CH2)2O)xNH2, —((CH2)2O)xCO2H, —NH(CH2)xNH2, —NH2, —NH(CH2)xOH, —NH(CH2)xCO2H, —CH3, or —CH2CH3; x=1, 2, 3, 4, 5, 6, 7, or 8.
R2=H, Cl, F, Br, I, —HC═O, —CO2H, —OH, ═O, —NH2, —RC═O, —CH(CH3)2, —CH3S═O, —CH3SO2, —CH═O, —CF3, —CH3, —CBr3, —CI3, CCl3, —SO2H, —SO2CH3, —SCH3, or —CH2CH3;
R3=H, Cl, F, Br, I, —HC═O, —CO2H, —OH, ═O, —NH2, —RC═O, —CH(CH3)2, —CH3S═O, —CH3SO2, —CH═O, —CF3, —CH3, —CBr3, —CI3, CCl3, —SO2H, —SO2CH3, —SCH3, or —CH2CH3;
R4=H, Cl, F, Br, I, —HC═O, —CO2H, —OH, ═O, —NH2, —RC═O, —CH(CH3)2, —CH3S═O, —CH3SO2, —CH═O, —CF3, —CH3, —CBr3, —CI3, CCl3, —SO2H, —SO2CH3, —SCH3, or —CH2CH3;
R5=H, Cl, F, Br, I, —HC═O, —CO2H, —OH, ═O, —NH2, —RC═O, —CH(CH3)2, —CH3S═O, —CH3SO2, —CH═O, —CF3, —CH3, —CBr3, —CI3, CCl3, —SO2H, —SO2CH3, —SCH3, or —CH2CH3.

Examples of Compounds Having Core Structure 2:

ZINC codes: 22594527 and 03883033.

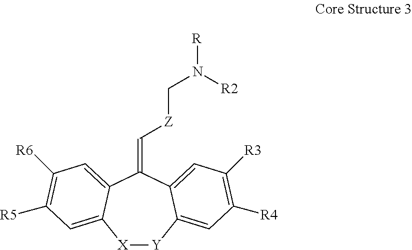

wherein

### X=C, S, N, O, or S═O;

### Y=C, S, N, O, or S=0;

R=—H, Cl, F, Br, I, —HC═O, —CO2H, —OH, ═O, —NH2, —RC═O, —(CH2)xOH, —(CH2)xNH2, —(CH2)xCO2H, —((CH2)xO)xNH2, —((CH2)xO)xCO2H, —((CH2)xO)xOH, —CHOCH3, —SO2H, —SO2CH3, —CH2SO2H, —CH2SO2CH3, —CH3, or —CH2CH3; x=1, 2, 3, 4, 5, 6, 7, or 8.
R2=H, Cl, F, Br, I, —HC═O, —CO2H, —OH, ═O, —NH2, —RC═O, —(CH2)xOH, —(CH2)xNH2, —(CH2)xCO2H, —((CH2)xO)xNH2, —((CH2)xO)xCO2H, —((CH2)xO)xOH, —CHOCH3, —SO2H, —SO2CH3, —CH2SO2H, —CH2SO2CH3, —CH3, or —CH2CH3; x=1, 2, 3, 4, 5, 6, 7, or 8.

### R3=H, Cl, F, Br, I, —HC═O, —CO2H, —OH, ═O, —NH2, —RC═O, —SO2H, —SCH3, —CH3, —CH2CH3;

R4=H, Cl, F, Br, I, —HC═O, —CO2H, —OH, ═O, —NH2, —RC═O, —CH(CH3)2, —CH3S═O, —CH3SO2, —CH═O, —CF3, —CH3, —CBr3, —CI3, CCl3, —SO2H, —SO2CH3, —SCH3, or —CH2CH3;
R5=H, Cl, F, Br, I, —HC═O, —CO2H, —OH, ═O, —NH2, —RC═O, —CH(CH3)2, —CH3S═O, —CH3SO2, —CH═O, —CF3, —CH3, —CBr3, —CI3, CCl3, —SO2H, —SO2CH3, —SCH3, or —CH2CH3;

### R6=H, Cl, F, Br, I, —HC═O, —CO2H, —OH, ═O, —NH2, —RC═O, —SO2H, —SCH3, —CH3, or —CH2CH3.

Example of Compound Having Core Structure 3:

ZINC code 00968257.

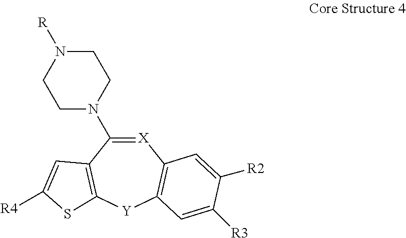

wherein

### X=C, S, N, or O;

### Y=C, S, N, O, —S═O, —SO2H, or —SO2CH3;

R=—H, —CH2CH2OH, —CH2CH2OCH2CH2OH, —CH═O, —(CH2)xNH2, —(CH2)xCO2H, —((CH2)2O)xNH2, —((CH2)2O)xCO2H, —NH(CH2)xNH2, —CH3, or —CH2CH3; x=1, 2, 3, 4, 5, 6, 7, or 8.
R2=H, Cl, F, Br, I, —HC═O, —CO2H, —OH, ═O, —NH2, —RC═O, —CH(CH3)2, —CH3S═O, —CH3SO2, —CH═O, —CF3, —CH3, —CBr3, —CI3, CCl3, —SO2H, —SO2CH3, —SCH3, or —CH2CH3;
R3=H, Cl, F, Br, I, —HC═O, —CO2H, —OH, ═O, —NH2, —RC═O, —CH(CH3)2, —CH3S═O, —CH3SO2, —CH═O, —CF3, —CH3, —CBr3, —CI3, CCl3, —SO2H, —SO2CH3, —SCH3, or —CH2CH3;
R4=H, Cl, F, Br, I, —HC═O, —CO2H, —OH, ═O, —NH2, —RC═O, —CH(CH3)2, —CH3S═O, or —CH3SO2, —CH═O, —CF3, —CH3, —CBr3, —CI3, CCl3, —SO2H, —SO2CH3, —SCH3, or —CH2CH3;

Example of Compound Having Core Structure 4:

ZINC code 52957434

Analogs of Ligand 281816 that are Predicted by AutoDock to Bind to HCV E2 as Well as or Better (i.e., they have an Equivalent or Lower Free Energy of Binding) than 281816.

| | |
|---|---|
| ZINC0000931 Amoxapine C1CN(CCN1)C2═NC3═CC═CC═C3OC4═C2C═C(C═C4)Cl | 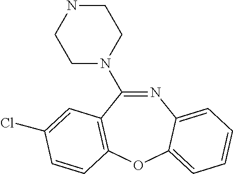 [SMILES: Clc1ccc2c(c1)C(N1CCNCC1)=Nc1ccccc1O2] |
| ZINC0001639 Loxapine CN1CCN(CC1)C2═NC3═CC═CC═C3OC4═C2C═C(C═C4)Cl | 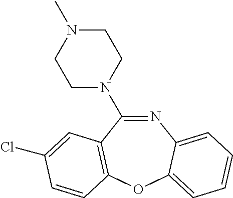 [SMILES: CN1CCN(C2=Nc3ccccc3Oc3ccc(Cl)cc32)CC1] |
| ZINC19632628 Quetiapine C1CN(CCN1CCOCCO)C2═NC3═CC═CC═C3SC4═CC═CC═C42 | 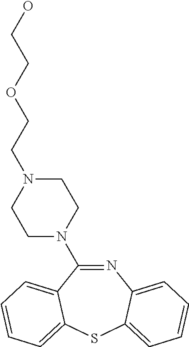 [SMILES: OCCOCCN1CCN(C2=Nc3ccccc3Sc3ccccc32)CC1] |
| ZINC00968257 Amitriptyline CN(C)CCC═C1C2═CC═CC═C2CCC3═CC═CC═C31 | 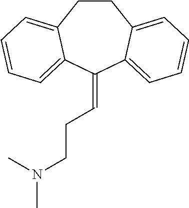 [SMILES: CN(C)CCC=C1c2ccccc2CCc2ccccc21] |
| ZINC19802239 1-[(5R)-9-fluoro-3-methylsulfanyl-5,6- dihydrobenzo[b][1]benzothiepin-5-yl]-4-methylpiperazin-4-ium C[NH+]1CCN(CC1)C2CC3═C(C═C(C═C3)F)SC4═C2C═C(C═C4)SC | 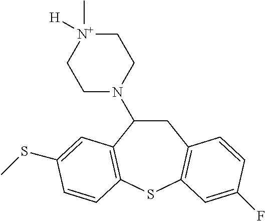 [SMILES: CSc1ccc2c(c1)C(N1CC[NH+](C)CC1)Cc1ccc(F)cc1S2] |
| ZINC22594527 N′-(3-methylsulfanyl-5,6-dihydrobenzo[b][1]benzothiepin-5- yl)ethane-1,2-diamine CSC1═CC2═C(C═C1)SC3═CC═CC═C3CC2NCCN | 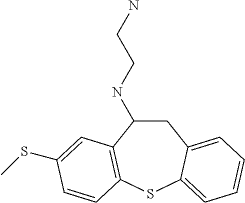 [SMILES: CSc1ccc2c(c1)C(NCCN)Cc1ccccc1S2] |
| ZINC19802414 1-methyl-4-[(5R)-2-(trifluoromethyl)-5,6- dihydrobenzo[b][1]benzothiepin-5-yl)piperazin-1-ium C[NH+]1CCN(CC1)C2CC3═CC═CC═C3SC4═C2C═CC(═C4)C(F)(F)F | 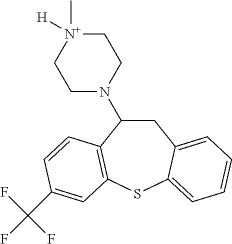 [SMILES: C[NH+]1CCN(C2Cc3ccccc3Sc3cc(C(F)(F)F)ccc32)CC1] |
| ZINC19802374 (5R)-5-(4-methylpiperazin-1-yl)-5,6- dihydrobenzo[b][1]benzothiepine-3-carbaldehyde O═Cc4cc2c(Sc1ccccc1CC2N3CCN(C)CC3)cc4 | 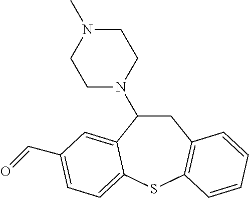 [SMILES: CN1CCN(C2Cc3ccccc3Sc3ccc(C=O)cc32)CC1] |
| Z1NC31554426 4-(5,6-dihydrobenzo[b][1]benzothiepin-5-yl)piperazine-1- carbaldehyde O═CN4CCN(C2c3c(Sc1ccccc1C2)cccc3)CC4 | 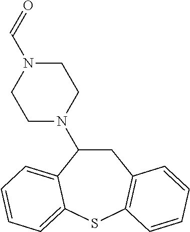 [SMILES: O=CN1CCN(C2Cc3ccccc3Sc3ccccc32)CC1] |
| ZINC19369143 1-[6-(4-methylpiperazin-1-yl)-5,6- dihydrobenzo[b][1]benzothiepin-3-yl]ethanone O═C(c4ccc3Sc1c(cccc1)C(N2CCN(C)CC2)Cc3c4)C | 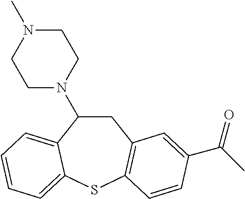 [SMILES: CC(=O)c1ccc2c(c1)CC(N1CCN(C)CC1)c1ccccc1S2] |
| ZINC22859648 (5R)-3-chloro-5-(4-methylpiperazin-1-yl)-5,6- dihydrobenzo[b][1]benzothiepin-9-ol Clc4cc2c(Sc1cc(O)ccc1CC2N3CCN(C)CC3)cc4 | 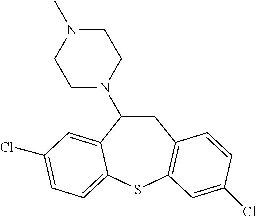 [SMILES: CN1CCN(C2Cc3ccc(Cl)cc3Sc3ccc(Cl)cc32)CC1] |
| ZINC33754515 3-[4-(3-methylsulfinyl-11-oxo-5,6- dihydrobenzo[b][1]benzothiepin-5-yl)piperazin-1-yl]propan-1-ol O═S(c1cc3c(cc1)S(═O)c2ccccc2CC3N4CCN(CCCO)CC4)C | 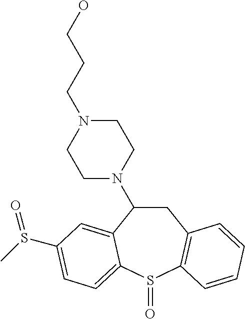 [SMILES: CS(=O)c1ccc2c(c1)C(N1CCN(CCCO)CC1)Cc1ccccc1S2=O] |
| ZINC19801738 2-[4-(2,8-dichloro-10,11-dihydrodibenzo[b,f]thiepin-10-yl)-1- piperazinyl]ethanol Clc4cc2c(Sc1ccc(Cl)cc1CC2N3CCN(CCO)CC3)cc4 | 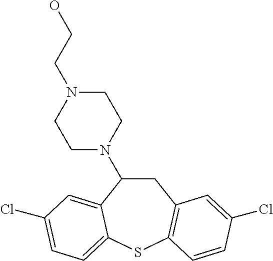 [SMILES: OCCN1CCN(C2Cc3cc(Cl)ccc3Sc3ccc(Cl)cc32)CC1] |
| ZINC33868652 3-[4-(3-methylsulfonyl-5,6-dihydrobenzo[b][1]benzothiopin-5- yl)piperazin-1-yl]propan-1-ol O═S(═O)(c4cc2c(Sc1ccccc1CC2N3CCN(CCCO)CC3)cc4)C | 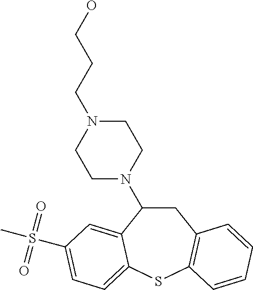 [SMILES: CS(=O)(=O)c1ccc2c(c1)C(N1CCN(CCCO)CC1)Cc1ccccc1S2] |
| ZINC65748577 Quetiapine dimer c1ccc2c(c1)C(═Nc3ccccc3S2)N4CCN(CC4)C5═Nc6ccccc6Sc7c5cccc7 | 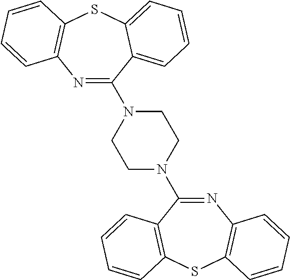 [SMILES: c1ccc2c(c1)N=C(N1CCN(C3=Nc4ccccc4Sc4ccccc43)CC1)c1ccccc1S2] |
| ZINC03883033 N1-(dibenzo[b,f][1,4]oxazepin-11-yl)ethane-1,2-diamine c1ccc2c(c1)C(═Nc3ccccc3O2)NCC[NH3+] | 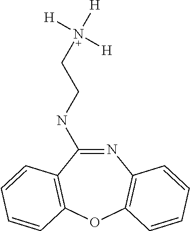 |
| ZINC19368911 Octoclothepine CN1CCN(CC1)C2CC3═CC═CC═C3SC4═C2C═C(C═C4)Cl | 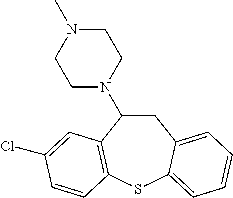 [SMILES: CN1CCN(C2Cc3ccccc3Sc3ccc(Cl)cc32)CC1] |
| ZINC52957434 Olanzapine or 2-methyl-4-(4-methyl-1-piperazinyl)-10H- thieno[2,3-b][1,5]benzodiazepine Cc1cc2c(s1)Nc3ccccc3N═C2N4CCN(CC4)C | 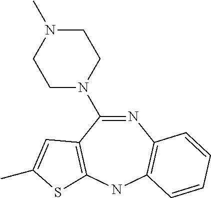 [SMILES: Cc1cc2c(s1)Nc1ccccc1N=C2N1CCN(C)CC1] |
| ZINC22459325 Isofloxythepin or 2-[4-(9-fluoro-3-propan-2-yl-5,6- dihydrobenzo[b][1]benzothiepin-5-yl)piperazin-1-yl]ethanol CC(C)C1═CC2═C(C═C1)SC3═C(CC2N4CCN(CC4)CCO)C═CC(═C3)F | 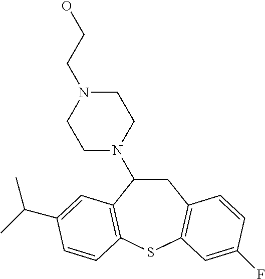 [SMILES: CC(C)c1ccc2c(c1)C(N1CCN(CCO)CC1)Cc1ccc(F)cc1S2] |
| ZINC26185346 Clothiapine or 8-chloro-6-(4-methylpiperazin-1- yl)benzo[b][1,4]benzothiazepine CN1CCN(CC1)C2═NC3═CC═CC═C3SC4═C2C═C(C═C4)Cl | 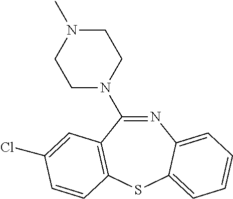 [SMILES: CN1CCN(C2=Nc3ccccc3Sc3ccc(Cl)cc32)CC1] |
| ZINC26247473 Fluperlapine or 3-fluoro-6-(4-methylpiperazin-1-yl)-11H- benzo[c][1]benzazepine CN1CCN(CC1)C2═NC3═C(CC4═CC═CC═C42)C═CC(═C3)F | 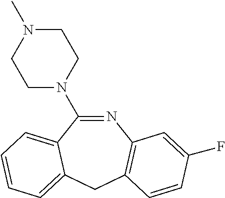 [SMILES: CN1CCN(C2=Nc3cc(F)ccc3Cc3ccccc32)CC1] |
| ZINC19796155 Clozapine or 8-Chloro-11-(4-methylpiperazin-1-yl)-5H- dibenzo[b,e][1,4]diazepine CN1CCN(CC1)C2═Nc3cc(ccc3Nc4c2cccc4)Cl | 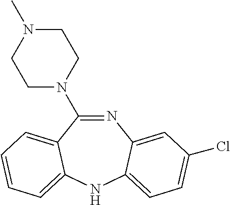 [SMILES: CN1CCN(C2=Nc3cc(Cl)ccc3Nc3ccccc32)CC1] |

### Structures of Other E2 Ligands

Examples of Compounds Having Core Structures that Differ from Core Structures 1, 2, 3 and 4:

1855333, 4428843, 1625746, 19325788, and 8652230.

| | |
|---|---|
| CI-670283 ZINC01855333 2,2′-spirobi[3,6,7,8-tetrahydro-1H-cyciopenta[g]naphthalene]- 5,5′-dione c1c2c(cc3c1CC4(C3)Cc5cc6c(cc5C4)C(═O)CCC6)C(═O)CCC2 | 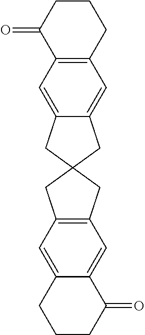 [SMILES: O=C1CCCc2cc3c(cc21)CC1(C3)Cc2cc3c(cc2C1)C(=O)CCC3] |
| NCI-86467 ZINC04428843 2-[[(8R,9S,13S,14S)-13-methyl-17-oxo-7,8,9,11,12,14,15,l6- octahydro-6H-cyclopenta[a]phcnanthren-3-yl]oxy]acetate C[C@]12CC[C@@H]3c4ccc(cc4CC[C@H]3[C@@H]1CCC2═O) OCC(═O)[O—] | 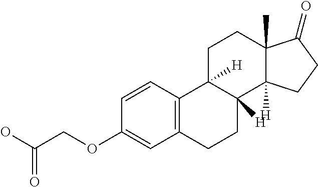 |
| NCI-639174 ZINC01625746 9-Glycineamido-20(S)-camptothecin•HCl CC[C@@]1(c2cc-3n(c(═O)c2COC1═O)Cc4c3nc5cccc(c5c4)NC(═O)C[NH3+])O | 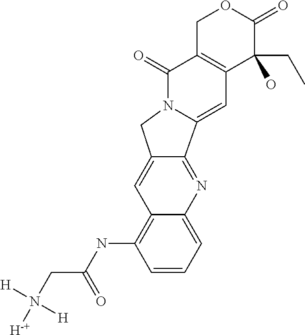 |
| NCI-81462 Z1NC19325788 PERHYDRO-1,4,7,9B-TETRAAZAPHENALENE C1CN[C@H]2CCN[C@H]3[NH+]2[C@H]1NCC3 | 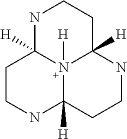 |
| NCI-403379 ZINC08652230 2-amino-3-(diaminomethylidene)quinazolin-3-ium-4-one c1ccc2c(c1)c(═O)n(c(n2)N)C(═[NH2+])N | 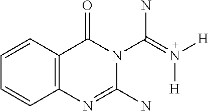 |

### HCV E2 Ligands Described by the Binding Sites on HCV E2.

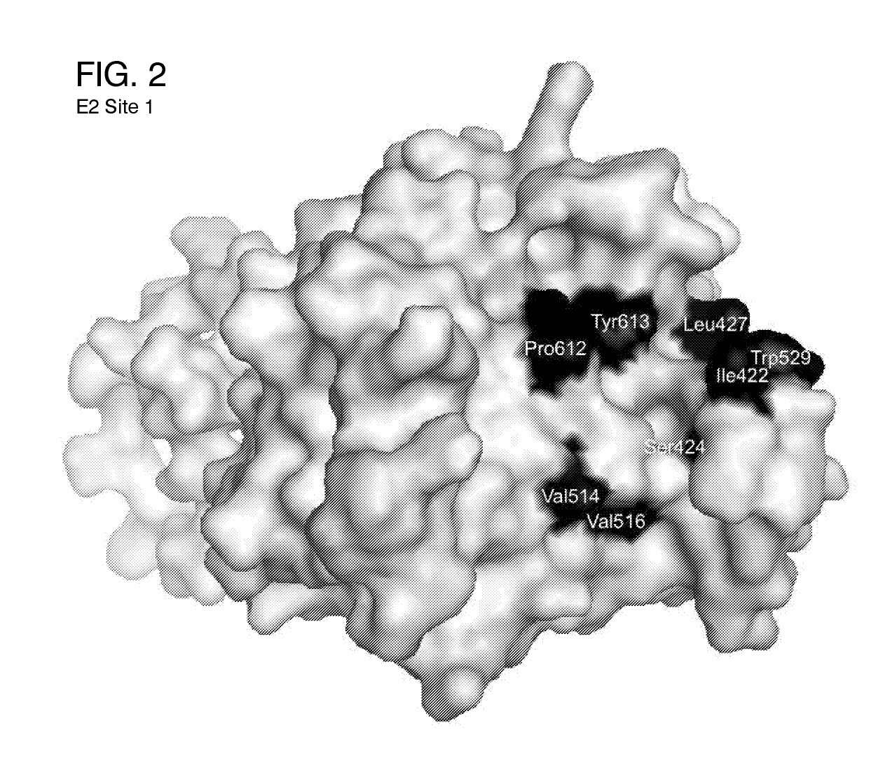

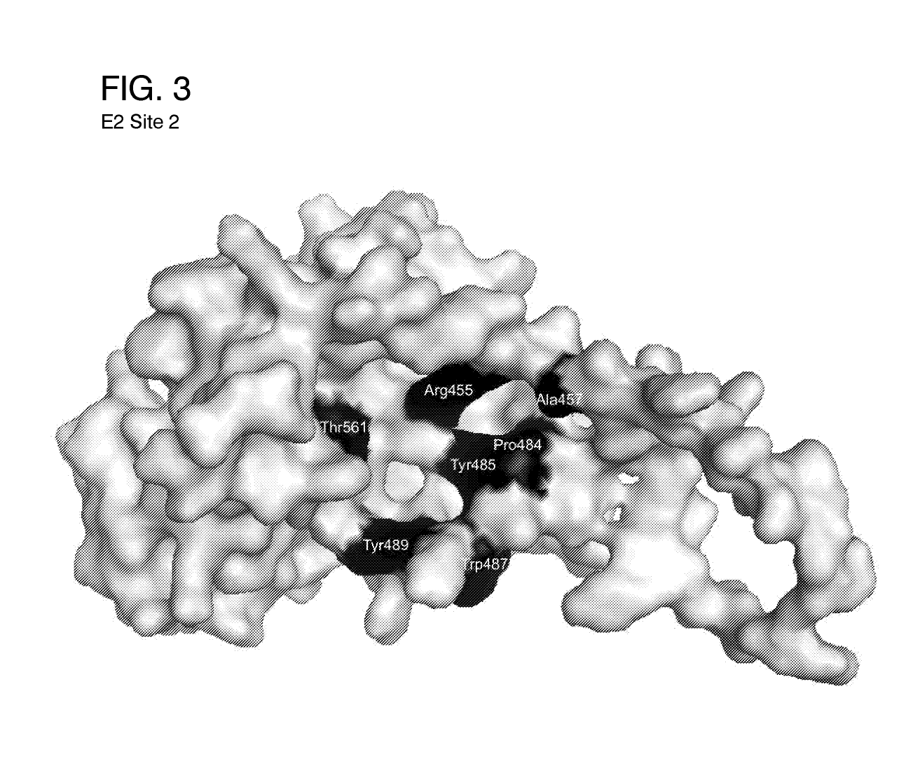

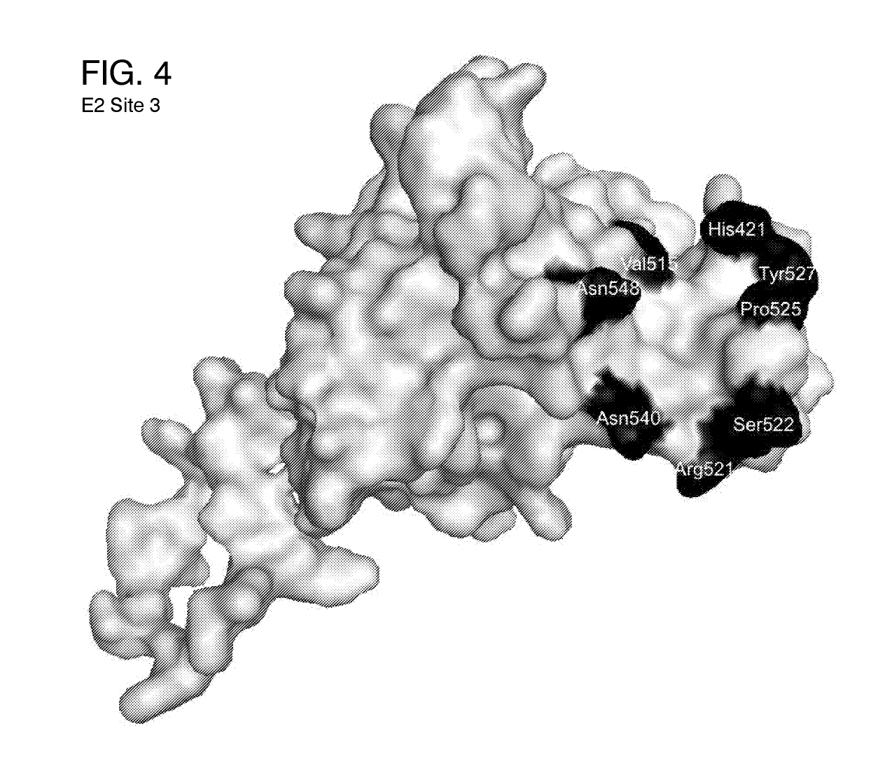

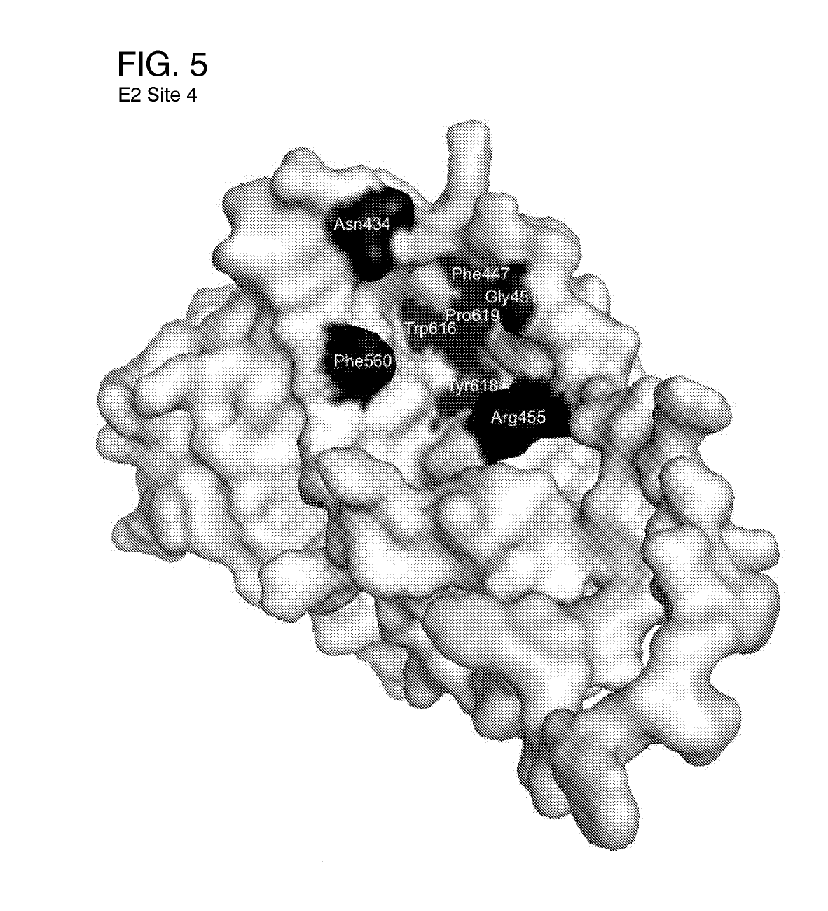

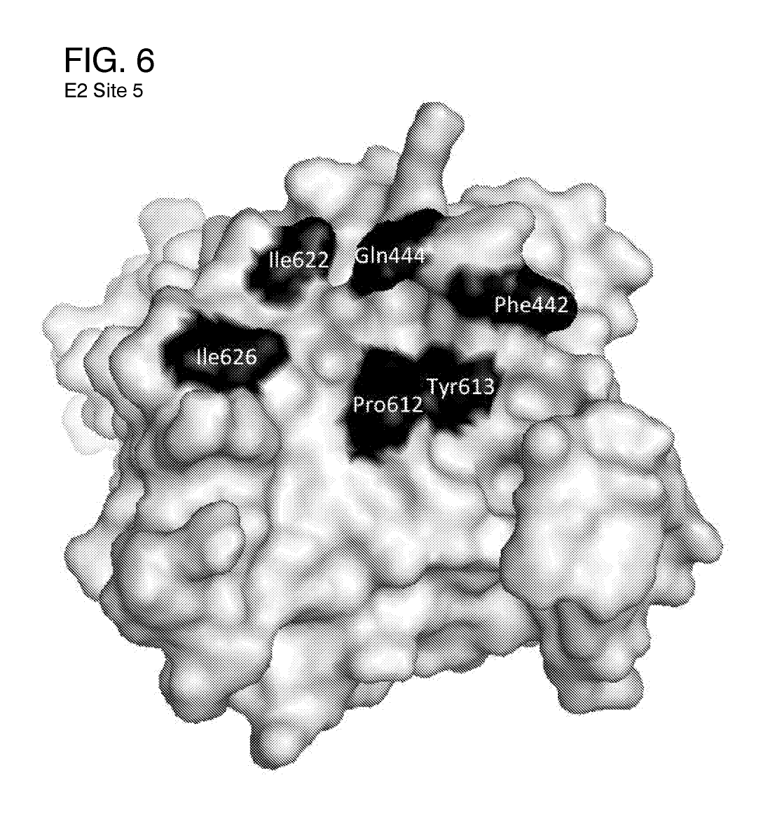

Ligands that bind to HCV E2 can also be described by their ability to bind within particular sites or to specific amino acid residues of the HCV E2 protein. The inventors have identified the following binding five sites on HCV E2 protein by the following amino acids that surround the bound ligand or are located within the sites where they bind, which are respectively shown in FIGS. 2-6.

Site 1: Pro612, Tyr613, Leu427, Trp529, Ile422, Ser424, Val514, Val516

Site 2: Arg455, Ala457, Trp487, Pro484, Tyr485, Tyr489, Thr561

Site 3: Tyr527, Pro525, Asn548, Asn540, His421, Arg521, Ser522, Val515

Site 4: Phe560, Asn434, Phe447, Tyr618, Pro619, Gly451, Arg455, Trp616

Site 5: Pro612, Tyr613, Gln444, Ile626, Ile622, Phe442

Small molecule ligands that bind to four, five, six or more amino acid residues of the sites recognized by the small molecule ligands described herein are also contemplated.

Multimers and Conjugates of Small Molecule HCV E2 Ligands

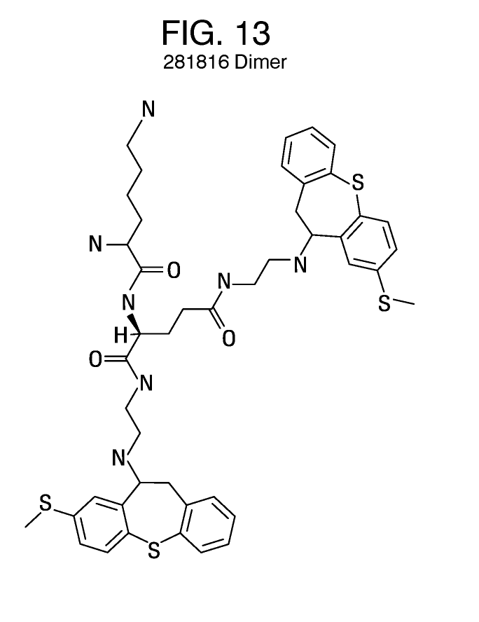

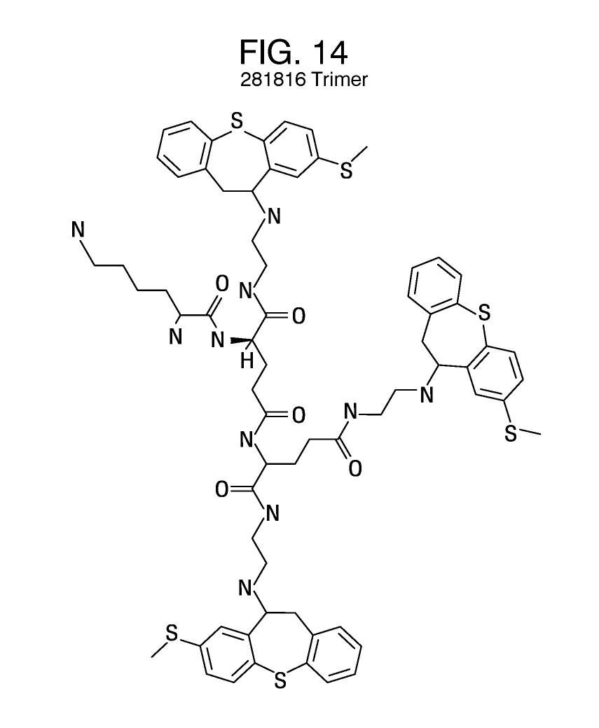

Multimeric forms of 281816 produced by linking two or more of the 281816 molecules together. Two specific examples are provided of a dimer (FIG. 13) and trimer (FIG. 14). Only one of the many types of linkers that can be used to create these multimers is shown. The exemplary linker used in these examples is the amino acid lysine. By linking pairs and triplets of molecules together, we and others have shown the binding affinity of the ligand conjugate for the protein can be increased 100 to 1 million fold. Linking together ligands not only improves the binding affinity of the ligand, but it will also improve its 1050 for inhibition of HCV infection.

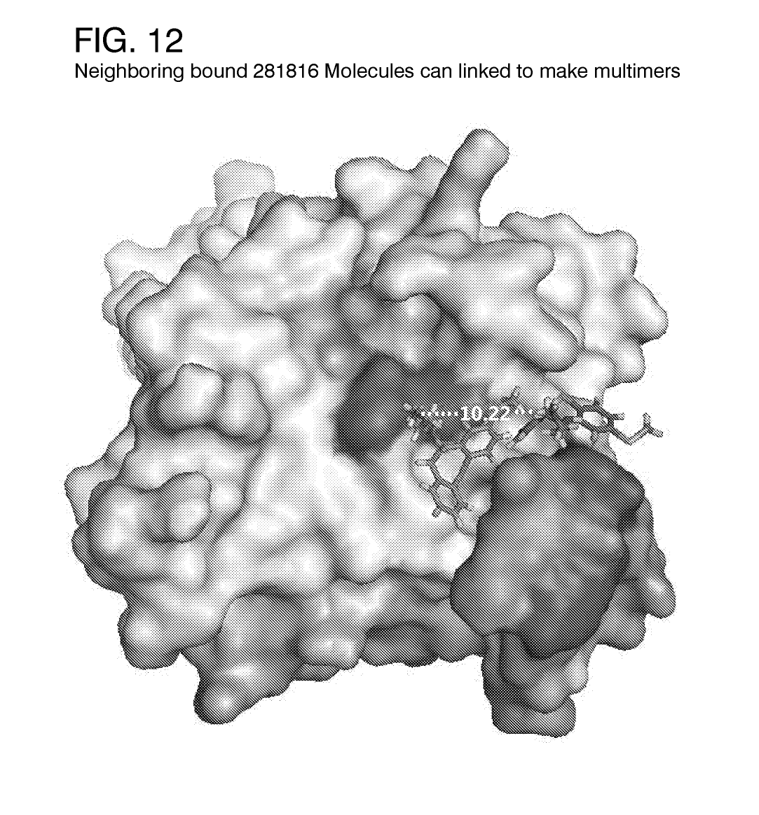

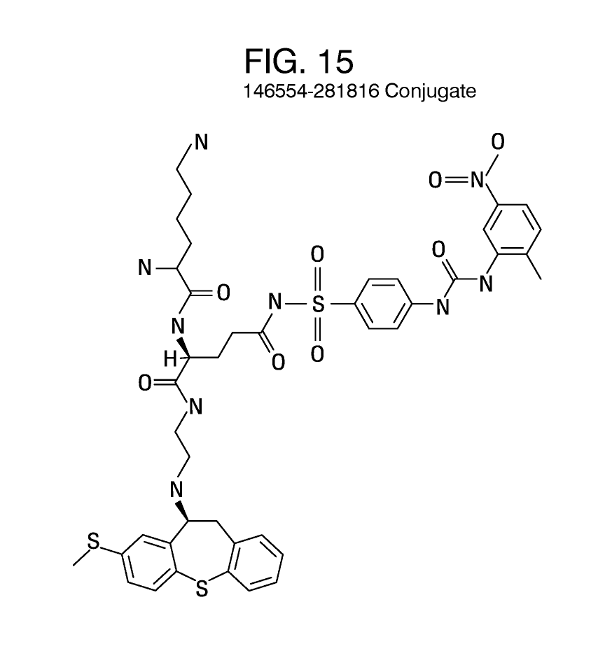

Conjugates of two or more ligands produced by linking 281816 with one or more other molecules that bind to neighboring, but different, sites on the E2 protein's surface can also be created to increase the ligands affinity, 1050 and selectivity for E2. An example of a 281816-146554 dimer is provided (FIG. 15). Multimers similar to this 281816-146554 dimer can be made by linking together any of the other 23 molecules identified to bind to E2, either as homomultimers or heteromultimers. This is possible because docking results indicate two or more of the same molecules, and different molecules, bind adjacent to each other in or around the same site; see FIG. 12 for one example.

A conjugate comprising a ligand that binds to HCV E2 protein and a ligand that binds to CD81, optionally where the ligand to HCV E2 binds with higher affinity to HCV E2 than that ligand for CD81 binds to CD81. Such conjugates provide a method for increasing the local concentration of a ligand to HCV E2 on or around cells that express CD81 comprising contacting it with a conjugate comprising a ligand for HCV E2 and a ligand for CD81. Representative ligands that bind to CD81 are disclosed by PCT/US2013/071056, filed Nov. 20, 2013, which is incorporated by reference. These ligands that bind CD81 include the following ligands that bind to CD81 identified by their numbers in the NCI Compound Diversity II Database:

| Site 1 | Site 2 | Site 3 | Site 4 | Site 5 |
|---|---|---|---|---|
| 165665 | 38743 | 93033 | 16631 | 68982 |
| 164965 | 156957 | 80807 | 68971 | 75866 |
| 689002 | 127947 | 25368 | 78623 | 90444 |
| 30930 | 73735 | 16162 | 81750 | 148832 |
| 5069 | 55573 | 25678 | 401077 | 601359 |
| 7436 | 41066 | 60239 | 408734 | 142446 |
| 21034 | 11891 | 75866 | 303800 | |
| 98026 | 63865 | 87504 | 75846 | |
| 123115 | 408860 | 89720 | 638134 | |
| 7962 | 362639 | 215276 | 70980 | |
| 16646 | 36914 | 331931 | 90444 | |
| 106863 | 20586 | 20586 | 89720 | |
| 117922 | 23895 | 403374 | 25678 | |
| 120631 | 252359 | 8481 | 215276 | |
| 7962 | 403374 | 5856 | 16162 | |
| 117922 | | | 60239 | |
| 106863 | | | | |
| 23895 | | | | |
| 120631 | | | | |
| 16646 | | | | |
| 252359 | | | | |
| 134137 | | | | |
| 97538 | | | | |
| 94914 | | | | |
| 31712 | | | | |
| 73170 | | | | |
| 144958 | | | | |
| 153172 | | | | |

### TERMS USED IN THIS DISCLOSURE

The term “HCV E2” is given its customary meaning. The invention contemplates variants of the HCV E2 protein from different strains of HCV; analogs of this protein from other viruses or microorganisms, especially analogs of segments of the protein that interact with CD81, or other natural or engineered forms of the HCV E2 protein or its variants or analogs. These variants, analogs or forms of the E2 protein can be characterized by a degree known HCV E2 sequence. Related or similar viruses may be identified based on their expression of a protein having these degrees of similarity or identity to Hepatitis C virus E2 protein. SEQ ID NOs: 1 and 5-14 describe HCV E2 amino acid sequences.

The term “CD81” is given its ordinary meaning in the art (Cluster of Differentiation-81). Human CD81 has been sequenced and is crystal structure determined. CD81 analogs from non-human animals are known and natural or artificial variants of CD81 are also contemplated. These are characterized by a degree of similarity or sequence identity to human CD81, for example, by a degree of similarity or identity of 80%, 85%, 87.5%, 90%, 92.5%, 95%, 97.5%, 98%, 99% to a known CD81 sequence. SEQ ID NOS: 2-4 describe CD81 amino acid sequences.

BLASTP may be used to identify an amino acid sequence having at least 80%, 85%, 87.5%, 90%, 92.5%, 95%, 97.5%, 98%, 99% sequence similarity or identity to a reference amino acid sequence using a similarity matrix such as BLOSUM45, BLOSUM62 or BLOSUM80. Unless otherwise indicated a similarity score will be based on use of BLOSUM80 which can be used to compare closely related CD81 or HCV E2 sequences. When BLASTP is used, the percent similarity is based on the BLASTP positives score and the percent sequence identity is based on the BLASTP identities score. BLASTP “Identities” shows the number and fraction of total residues in the high scoring sequence pairs which are identical; and BLASTP “Positives” shows the number and fraction of residues for which the alignment scores have positive values and which are similar to each other. Amino acid sequences having these degrees of identity or similarity or any intermediate degree of identity of similarity to the amino acid sequences disclosed herein are contemplated and encompassed by this disclosure.

A “small organic molecule” includes low molecular weight organic compounds or approximately 800 daltons or less that are not polymers. Small molecules according to the invention will bind to HCV E2. These small molecules may bind to a particular site on HCV E2, such as Site 1, 2, 3, 4 or 5 identified by the inventors, or to more than one site. These ligands may have a greater or lesser affinity for HCV E2 than a natural ligand from HCV E2, such as the CD81 protein. Ligand binding can passively block binding of other ligands to HCV E2 and/or trigger or transduce allosteric effects in HCV E2, for example, a ligand that binds to HCV E2 can modulate, inhibit, or block its binding to CD81 and thus inhibit infection of CD81-bearing cells.

The binding affinity and ligand efficacy of a HCV E2 ligand molecule can be determined by methods known in the art. Different ligands will exhibit different binding affinities for sites on HCV E2, for example, binding affinity can range from 1 nM to 10,000 nM and all intermediate values within this range, such as 1 nM, 10 nM, 100 nM, 1,000 nM, 5,000 nM and 10,000 nM. The inventors have found that ligands or ligand conjugates that bind to at least two of Sites 1, 2, 3, 4 or 5, identified on HCV E2, bind more strongly to HCV E2 than individual ligands for each site.

The invention contemplates such small molecules per se, as well as larger conjugates or hybrid molecules containing one or more small molecules that interact with HCV E2. The larger conjugates or hybrid molecules may comprise more than one determinant that binds to HCV E2, more than one copy of a particular HCV E2-binding determinant, or determinants that bind to different sites on HCV E2.

Small organic molecules according to the invention are publicly available, for example, as described in the ZINC database. ZINC is a free database of commercially-available compounds for virtual screening. ZINC contains over 21 million purchasable compounds in ready-to-dock, 3D formats. ZINC is provided by the Shoichet Laboratory in the Department of Pharmaceutical Chemistry at the University of California, San Francisco (UCSF), see: Irwin, Sterling, Mysinger, Bolstad and Coleman, J. Chem. Inf. Model. 2012DOI: 10.1021/ci3001277. The original publication is Irwin and Shoichet, J. Chem. Inf. Model. 2005; 45(1):177-82PDF, DOI. The compounds described in the ZINC database as of Feb. 25, 2014 are incorporated by reference to the Zinc database or to the publications above.

Functional variants of the small organic molecules of the invention are also contemplated. Like the unmodified small organic molecule, these variants will bind to HCV E2 but may have one or more substitutions to the chemical structure of the unmodified small organic molecule ligand. Substitutions to the core structure of a small organic molecule ligand described herein may include functional groups that improve binding to HCV E2, confer specific properties such as solubility or stability, or which affect the pharmacokinetics, biodistribution, absorption, tissue uptake, residence time in tissue, or that minimize toxicity, excretion or metabolism; that enable the small molecule ligand to be conjugated to other molecules; or that facilitate the diagnostic use of the small molecule ligand.

Examples include the addition or substitution to a ring or other structural element with other atoms such as at least 1, 2, 3, 4, 5 or 6 hydrogen atoms, halogens (chlorine, fluorine, iodine, bromine), functional groups such as carboxylic, amino, amine, amide, azo, ester, thiol, sulfonyl, nitro, alkoxy, acetyl, acetoxy, hydroxyl or other alcohol, aldehyde, carbonyl, alkyl, alkene or alkyne groups or chains, ether, epoxide, hydrazone, imide, imine, isocyanate, isonitrile, isothiocyanate, ketone, nitrile, nitrene, nitro, nitroso, organophosphorus, oxime, phosphonic or phosphonous acid, sulfone, sulfonic acid, sulfoxide, thiocyanate, thioester, thioether, thioketone, urea, pyridine groups or other aromatic rings. Other substituents may include metals or radioisotopes (to enable detection or visualization), tags such as fluorescent dyes or molecules, biotin, digoxigenin, peptides, amino acids (to improve uptake, delivery and biodistribution).

The molecules, including multimers and conjugates, of the invention may be compounded as salts. Examples of pharmaceutically acceptable salts include, but are not limited to, salts prepared from pharmaceutically acceptable acids or bases, including organic and inorganic acids and bases. When the preferred form of the active compound for use is basic, salts may be prepared from pharmaceutically acceptable acids. Suitable pharmaceutically acceptable acids include acetic, benzenesulfonic (besylate), benzoic, p-bromophenylsulfonic, camphorsulfonic, carbonic, citric, ethanesulfonic, fumaric, gluconic, glutamic, hydrobromic, hydrochloric, hydroiodic, isethionic, lactic, maleic, malic, mandelic, methanesulfonic (mesylate), mucic, nitric, oxalic, pamoic, pantothenic, phosphoric, succinic, sulfuric, tartaric, ptoluenesulfonic, and the like. Examples of such pharmaceutically acceptable salts include, but are not limited to acetate, benzoate, hydroxybutyrate, bisulfate, bisulfite, bromide, butyne-1,4-dioate, carpoate, chloride, chlorobenzoate, citrate, dihydrogenphosphate, dinitrobenzoate, fumarate, glycollate, heptanoate, hexyne-1,6-dioate, hydroxybenzoate, iodide, lactate, maleate, malonate, mandelate, metaphosphate, methanesulfonate, methoxybenzoate, methylbenzoate, monohydrogenphosphate, naphthalene-1-sulfonate, naphthalene-2-sulfonate, oxalate, phenylbutyrate, phenylproionate, phosphate, phthalate, phylacetate, propanesulfonate, propiolate, propionate, pyrophosphate, pyrosulfate, sebacate, suberate, succinate, sulfate, sulfite, sulfonate, tartrate, xylenesulfonate, and the like. In addition to salts, acidic or basic forms of these molecules may also be prepared by selection of an appropriate pH.

Linkers or Spacers. In some embodiments of the invention linkers or spacers are used. These linkers or spacers may be used to join small molecules that bind to different portions of HCV E2 and to space the small molecule moieties in a joined molecule so that they can bind to different parts of HCV E2. For example, a small molecule that binds to a first site on HCV E2 may be spaced from 0 (e.g., where a carboxyl group on one small second or subsequent site using a linker of an appropriate length. In most cases, linkers would range from 2 or 3 to about 7-10 Å. Generally, small organic ligand molecules will be joined by linkage to a single position on each ligand to another ligand or to an intervening linker. However, linkage may also occur at 2 or more positions on a ligand molecule to another ligand molecule or linker. Linkers may have different chemical structures including straight-chain and branched chain structures, and structures including saturated or unsaturated bonds (e.g., alkyl, alkenyl or alkynyl), heteroatoms (e.g., nitrogen, oxygen or sulfur) or aromatic moieties. Bivalent and multivalent linkers may contain the same or different reactive chemical groups for linking two or more small molecule ligands for HCV E2. Linkers may range from 1, 2, 3, 4, 5, 6, 7, 8, 9, 10, 11, 12, 13, 14, 15 or more atoms in length. HCV E2 ligands where each ligand has a chemical group that can react with a chemical group on another ligand.

Linkers suitable for use in the invention are known in the art and are incorporated by reference to Ducry, et al., Bioconjugate Chem. 21, 5-13, Antibody-Drug Conjugates: Linking Cytotoxic Payloads to Monoclonal Antibodies (2010); to Gordon, et al., J. Chem. Technol. Biotechnol. 74:835-851, Solid phase synthesis—designer linkers for combinatorial chemistry: a review (1999), and to Leitner, et al., Mol. Cell. Proteonom. 9:1634-1649 (2010), which are incorporated by reference. Exemplary linkers include lysine, glutamic acid and polyethylene glycol (PEG) moieties.

Generally, the small molecule ligands of the invention are not polymers. However, conjugates of small molecule ligands may contain multiple units of one or more small organic molecule ligands, for example, as linked to a dendrimer. In addition to small organic molecules linked together with a chemical linker, these small organic molecule ligands may be conjugated to larger moieties such as antibodies and other proteins, nucleic acids and nucleic acid analogs, carbohydrate and sugar molecules, etc. The small molecule ligands, conjugates or hybrids may also be conjugated to detectable moieties such as avidin or streptavidin, biotin or other detectable tags.

Hybrid molecules that comprise chemical moieties from two or more known small organic molecule ligands are engineered by a process of fragment-based extension.

A “composition” or “pharmaceutical or therapeutic composition” according to the invention refers to a combination of carrier, excipient, or solution with a small molecule, ligand conjugate or hybrid molecule. The term “pharmaceutically acceptable carrier” includes any and all carriers and excipients such as diluents, solvents, dispersing agents, emulsions, lipid bilayers, liposomes, coatings, preservatives including antibacterial or antifungal agents, isotonic agents, pH buffers, and absorption modulating agents, and the like, compatible with the molecules of the present invention and suitable for pharmaceutical administration. The use of such carriers, disintegrants, excipients and agents for administration of pharmaceutically active substances is well known in the art, see the Handbook of Pharmaceutical Excipients, 3^{rd} edition, Am. Pharm. Assoc. (2000) which is incorporated by reference. The pharmaceutical compositions of the invention are generally formulated for compatibility with an intended route of administration, such as for parenteral, oral, or topical administration.

The therapeutic compositions of the invention include at least one molecule, multimer or conjugate according to the invention in combination with pharmaceutically acceptable carrier. A “pharmaceutically acceptable carrier” will be at least one component conventionally admixed with, and used for, the administration of an active ingredient, biological product, or drug. A therapeutic composition may be sterile or in a form suitable for administration to a human or non-human subject. A carrier may contain any pharmaceutical excipient used in the art and any form of vehicle for administration. The compositions may be, for example, injectable solutions, aqueous suspensions or solutions, non-aqueous suspensions or solutions, sprays, solid and liquid oral formulations, salves, gels, ointments, intradermal patches, creams, lotions, tablets, capsules, sustained release formulations, and the like. Additional excipients may include, for example, colorants, taste-masking agents, solubility aids, suspension agents, compressing agents, enteric coatings, sustained release aids, and the like.

A pharmaceutical composition according to the present invention can be prepared and administered in a wide variety of dosage forms. For example, it can be made in inert, pharmaceutically acceptable carriers which are either solid or liquid. Solid form preparation include powders, tablets, dispersible granules, capsules, cachets, and suppositories. Other solid and liquid form preparations could be made in accordance with known methods of the art. The quantity of active compound in a unit dose of preparation may be varied or adjusted depending upon a patient's particular condition and requirements. Factors such as the sex and age group of the patient, medical condition of the patient including the severity of nature of HCV infection or risk of infection, the intended use (chemoprophylaxis or treatment of HCV) and the identity of the particular active molecule, multimer, or conjugate and its dosage form may be taken into account. One may determine an effective dosage for inhibiting HCV infection using conventional methods. A suitable dosage form may be selected by one of skill in the art from forms such as those described by “Dosage Form”; NCI Thesaurus OID: 2.16.840.1.113883.3.26.1.1 NCI concept code for pharmaceutical dosage form: C42636; accessible at http://www.fda.gov/ForIndustry/DataStandards/StructuredProductLabeling/ucm162038.htm (last accessed Feb. 7, 2014) which is hereby incorporated by reference.

Orally administered compositions may include a solid carrier or excipient or can be formulated as liquid or gel preparations and may include an edible or inert carrier and may be enclosed in capsules, compressed into tablets, or formulated as a troche. Orally administered compositions may be prepared in a time-release or encapsulated form to prevent degradation in the stomach and optimize uptake of the active molecule, multimer or conjugate.

Injectable compositions may be formulated by methods well known in the art and may encompass sterile solutions or dispersions of therapeutic molecules. Such will usually include a sterile diluent, such as water, normal saline, or other buffer compatible with the molecules of the invention. Injectable compositions may be prepared in unit dosages or in unit dose containers, such as vials, ampules, or syringes.

Conventional buffers and isotonic agents may be used and pH may be adjusted using well known agents, such as HCl or NaOH or buffers. Antimicrobial or bacteriostatic agents, chelating agents, such as EDTA or EGTA, and antioxidants and preservatives may be present.

An antiseptic, disinfectant, or other virus-inhibitory composition, which need not be in a sterile or pharmaceutically acceptable form, may also be formulated to contain the HCV E2 binding ligands disclosed herein. It can be incorporated into a composition used to treat or clean materials that can come into contact with blood or other sources of HCV. These include surgical or medical tools, instruments or equipment, such needles and syringes; multiple-use medication vials; infusion bags; and improperly sterilized surgical equipment; tattooing or scarification equipment; knives or kitchen utensils, and weapons that contact blood or other HCV contaminants. It may also be incorporated into a composition used to clean or sterilize surfaces, such as a disinfectant, antiseptic, skin or hand cleaner, a wash or prophylactic, such as a mouthwash or dental rinse, or a moisturizer or prophylactic for a mucous membrane. For surgical, dental and other similar procedures it may be incorporated into a rinse, wash, adhesive, floss, pick, gauze, wrap, packing, bandage and the like.

The therapeutic compositions of the invention may be administered by any acceptable route of administration including topically, on to a mucous membrane, orally or enterically or parenterally. These routes include, but not limited to topical, transmucosal, orally (including buccal, sublingual), mucosally (conjunctiva, nasal, sinal, urethral, vaginal, intestinal, rectal), enteric, transdermal, intradermal, subcutaneous (s.c.), intramuscular, intraperitoneal, intravenous (i. v.) intracardiac, into a joint or bone, into an organ (brain, spinal cord, eye, ear, liver, spleen, kidney, gall bladder, bladder), into bone, cartilage, or joint tissue, by inhalation (e.g., intranasal, intratracheal, intrapulmonary, or intrabroncial), oral, subuccal. Routes may be selected by those of skill in the art from those listed in the U.S. FDA, CDER, Data Standards Manual “Routes of Administration”; FDA Data Element Number. None. CDER Data Element Number. C-DRG-00301; Data Element Name. Route of Administration; Data Element OID: 2.16.840.1.113883.3.26.1.1.1 Data Element NCI Concept ID: C38114; Version Number 004 accessible at http://_www.fda.gov/Drugs/DevelopmentApprovalProcess/FormsSubmissionRequirements/ElectronicSubmissions/DataStandardsManualmonographs/ucm071667.htm; (last accessed Feb. 21, 2014) which is hereby incorporated by reference.

### Conjugates of Ligands Binding to HCV E2 Protein and Ligands Binding to CD81

The following are examples of conjugates containing two ligands, one that binds to the E2 protein and one that binds to CD81. Such conjugates should improve the effectiveness of the drug by binding to CD81 and increasing the local concentration of the drug in the vicinity of CD81 on the cell surface. The examples contain ligand 281816 or one of its analogs that binds to HCV E2 and ligand 73735 that binds to CD81. This combination of ligands is particularly useful because 73735 has also been determined to bind to E2. Consequently, the concentration of the conjugate would be increased around CD81 by its binding through the 73735 ligand to CD81 and also around E2 by the 281816 ligand binding to E2 and also some of the conjugate binding to other sites on E2 where 73735 binds.

| | |
|---|---|
| Structure 1 Ligands: one 73735, two 281816 analogs No linker | 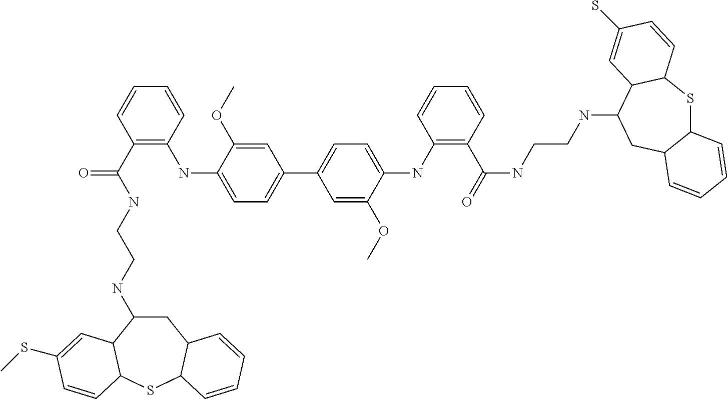 [SMILES: COc1cc(-c2ccc(Nc3ccccc3C(=O)NCCNC3CC4C=CC=CC4SC4C=CC(SC)=CC34)c(OC)c2)ccc1Nc1ccccc1C(=O)NCCNC1CC2C=CC=CC2SC2C=CC(S)=CC12] |
| Structure 2 Ligands: one 73735, one 281816 analog No linker | 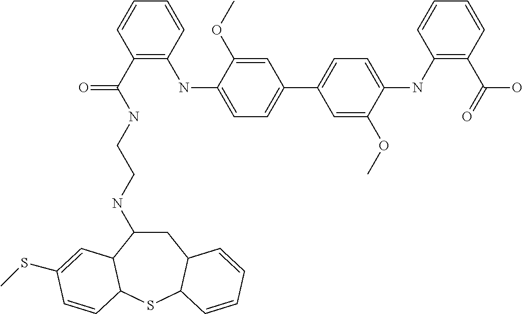 [SMILES: COc1cc(-c2ccc(Nc3ccccc3C(=O)NCCNC3CC4C=CC=CC4SC4C=CC(SC)=CC34)c(OC)c2)ccc1Nc1ccccc1C(=O)O] |
| Structure 3 Ligands: one 73735, two 281816 analogs No linker | 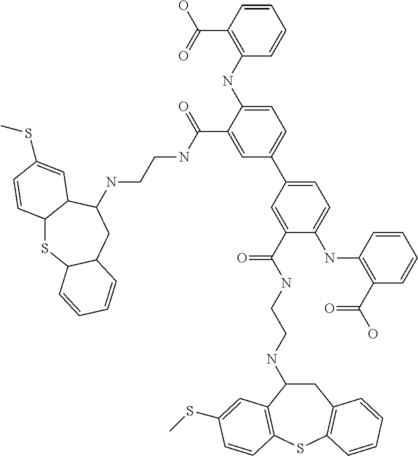 [SMILES: CSC1=CC2C(NCCNC(=O)c3cc(-c4ccc(Nc5ccccc5C(=O)O)c(C(=O)NCCNC5Cc6ccccc6Sc6ccc(SC)cc65)c4)ccc3Nc3ccccc3C(=O)O)CC3C=CC=CC3SC2C=C1] |
| Structure 4 Ligands: one 73735, one 281816 analog No linker | 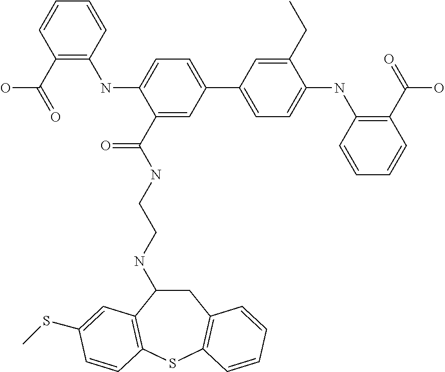 [SMILES: CCc1cc(-c2ccc(Nc3ccccc3C(=O)O)c(C(=O)NCCNC3Cc4ccccc4Sc4ccc(SC)cc43)c2)ccc1Nc1ccccc1C(=O)O] |
| Structure 5 Ligands: one 73735, one 281816 Lysine linker | 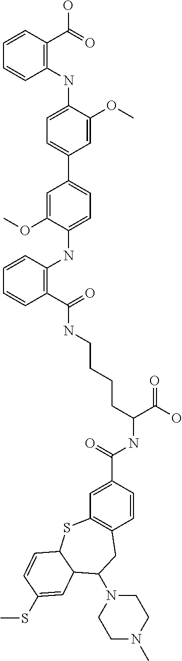 [SMILES: COc1cc(-c2ccc(Nc3ccccc3C(=O)NCCCCC(NC(=O)c3ccc4c(c3)SC3C=CC(SC)=CC3C(N3CCN(C)CC3)C4)C(=O)O)c(OC)c2)ccc1Nc1ccccc1C(=O)O] |

### EXAMPLES

### Example 1

### Creation of the Homology Model of E2 Used for Docking

A crystal structure of E2c deposited in the PDB under a code 4MWF was resolved by Kong et al. [12] at a resolution of 2.65 Angstroms. However, upon examination of the structure file prior to docking, the set of reported atom coordinates of the protein was found to be incomplete. In addition to the coordinate file containing structural information for only 171 residues out of the 363 amino acids present in the full-length protein, structural information was missing for several peptide segments or loops (P453-P491, V574-N577 and F586-R596) within the structural core of the protein. The crystal structure was also obtained using a protein sequence that contained several amino acids (S422, D423, S444, E445, and D448) that were not present in the genotype 1a sequence of the E2 protein.

In order to prepare a more complete version of the structure for docking, we have performed several homology modeling and structure analysis tasks using the coordinates of E2c as a template. The final structural model was created using the AS2TS system [21] based on atom coordinates from the PDB chains 4mwf C and 4mwf D. A structural search for similar fragments in proteins in the PDB that could be used to model missing loop regions was performed using the StralSV algorithm [22], which identifies protein structures that exhibit structural similarities despite low primary amino acid sequence similarity. Exhaustive structure similarity searches of 90 residue structural fragments of E2 conducted using the entire PDB database (255,302 PDB chains) revealed that no structural homologs could be found at the level of calculated structure similarities by LGA score [41] higher than LGA_S=45%. Thus, modeling the structure of the insertions needed to fill in missing regions in the experimentally solved crystal structure and to complete the model was not a trivial task. The side-chain prediction was accomplished using SCWRL [23] when residue-residue correspondences did not match. Residues that were identical in the template and E2 protein were copied from the template onto the model. Potential steric clashes were identified in the unrefined model using a contact-dot algorithm in the MolProbity software package [24], and the constructed model was finished with relaxation using UCSF Chimera [25].

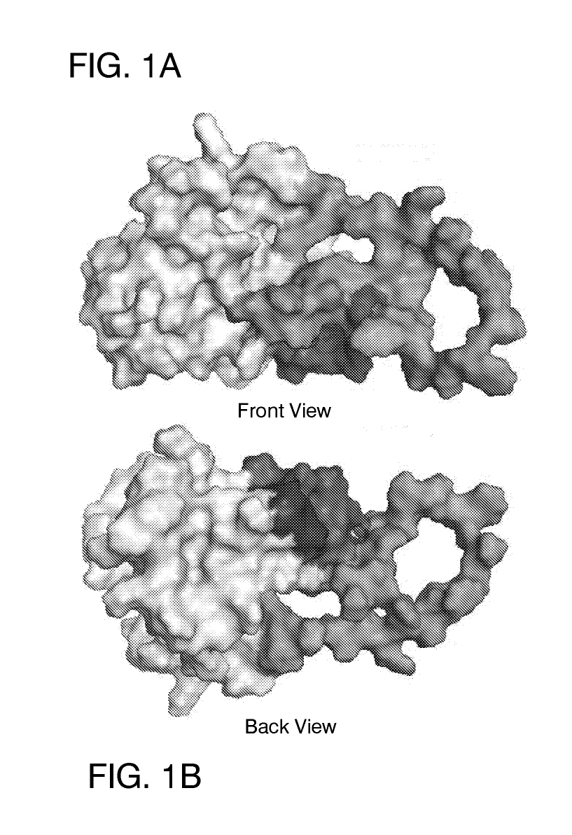

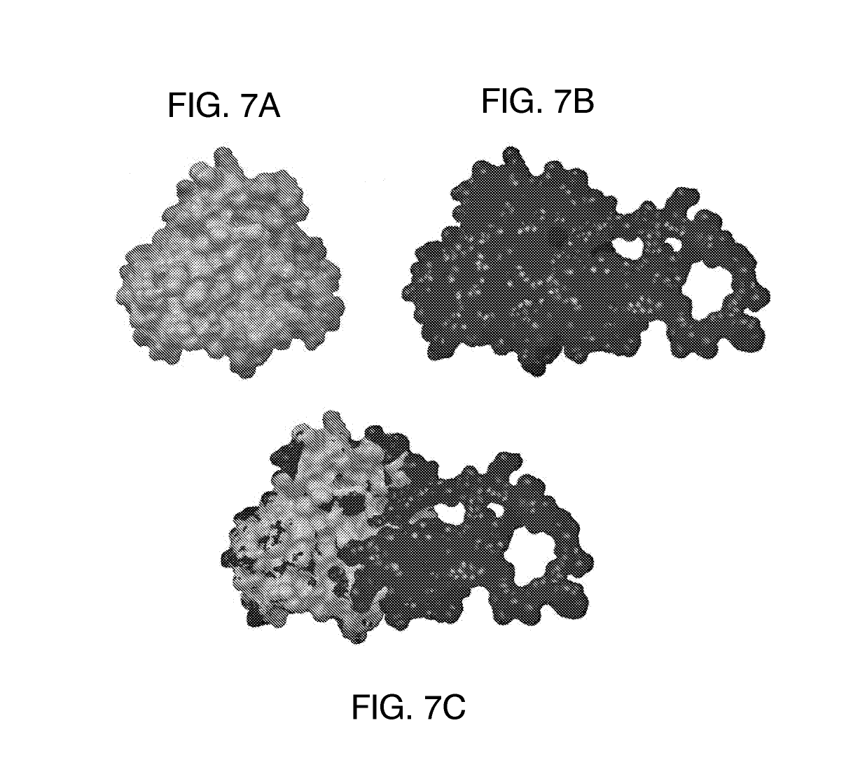

By applying a combination of structural modeling and analysis methods to the E2 crystal structure as described in the Materials and Methods section, we were able to construct a model that met all the requirements needed for docking. This model contains the peptide segments and loops that are missing in the E2c structure (FIG. 1), the domain containing a missing amino acid shown by alanine mutation to be critical for E2 binding (W487), as well as the correct sequence for the HCV genotype 1a E2 protein. Three regions that have been identified by others to be critical for E2 binding to CD81 are contained in the model in their entirety (FIG. 1). Currently, only three of the twenty-one Region 1 amino acids (H421-N423) are present in the model. A comparison of our model to the E2c structure (see superposition of two structures in FIG. 7) shows the main core regions are, as one would expect, very similar. The differences that are observed in the core region are small and appear to reflect minor differences caused by the removal of the four extra amino acids in the crystal structure's sequence. The large region that does differ corresponds to the missing peptide segments.

### Example 2

### Virtual Screen of the NCI Diversity Set III to the HCV E2 Protein Model

AutoDock VINA 1.1.2 (VINA) [26] was used to perform a Virtual Screen of the NCI Diversity Set III against the model of the E2 protein from Hepatitis C Virus (HCV), using the homology model that was created based on the new crystal structure developed by Kong et al. [12] (PDB ID: 4MWF.pdb). The model of the protein was prepared using the MolProbity Server (to add all of the hydrogen atoms and to flip the HIS/ASN/GLN residues if doing so significantly lowered the energy) and AutoDockTools4.2 (which added the Gasteiger-Marsili charges and merged the non-polar hydrogens onto their respective heavy atoms) [27,28]. The NCI Diversity Set III library containing 1,715 models of compounds was obtained from the ZINC server (http://zinc.docking.org) [29]. The multi-molecule “mol2” files from ZINC were prepared for docking calculations using Raccoon [30], which added the Gasteiger-Marsili charges, merged the non-polar hydrogen atoms onto their respective heavy atoms, and determined which bonds should be allowed to freely rotate during the calculations, to generate the “pdbqt” docking input format.

Four different, overlapping grid boxes were used in this virtual screen to enable the docking calculations to explore almost the entire surface of this E2 model (except for the large, flexible loop that was added to the model and the relatively flat surface near it). Since large grid boxes were used in these calculations, the “exhaustiveness” setting in VINA was increased to 20. Each calculation used 8 CPUs on the Linux cluster at Rutgers University-NJMS. The first box, which included P490, was centered at 38.829, 12.968, −40.958 (x, y, z) and had the following dimensions: 24.0×35.0×30.0 (x, y, z in Angstroms). The second grid box, which included G436, was centered at 48.401, 11.791, −14.449 (x, y, z) and had a size of: 32.0×36.0×24.0 (x, y, z in Angstroms). The third grid box, which included S528, was centered at 51.644, 25.877, −27.795 (x, y, z) and encompassed 30.0×30.0×30.0 Angstroms. The fourth grid box, which was selected to include the side of E2 not covered by the previous three grid boxes, was centered at 57.777, 12.968, −34.067 (x, y, z) and enclosed 24.0×35.0×32.0 Angstroms (x, y, z).

The docking outputs generated by VINA were processed and filtered using python scripts from Raccoon2 and Fox [30]. The top-ranked VINA mode from each docking calculation was harvested, and 17 different sets of energetic and interaction-based filters were investigated to harvest the most promising docking results for visual inspection. The following parameters were explored in the filtering process: −e indicates the minimum estimated Free Energy of Binding from the VINA score in kcal/mol, −l is the minimum ligand efficiency value in kcal/mol/heavy atom, −S is the minimum number of hydrogen bonds between the ligand and target, and —H indicates that the ligand had to form a hydrogen bond with either a backbone amino group (::N) or a backbone carbonyl oxygen (::O) of any residue in that grid box. These filters were investigated for the results from each of the four grid boxes:

1) −e −6.5 −1 −0.29 −S 3

2) −e −7.0 −1 −0.29 −S 3

3) −e −7.5 −1 −0.29 −S 3

4) −e −8.0 −1 −0.29 −S 3

5) −e −7.0 −1 −0.29 −S 4

6) −e −7.5 −1 −0.29 −S 4

7) −e −8.0 −1 −0.29 −S 4

8) −e −6.5 −1 −0.29 −S 3 —H ::N

9) −e −6.5 −1 −0.29 −S 3 —H ::O

10) −e −7.0 −1 −0.29 −S 3 —H ::N

11) −e −7.0 −1 −0.29 −S 3 —H ::O

12) −e −7.0 −1 −0.29 −S 4 —H ::N

13) −e −7.0 −1 −0.29 −S 4 —H ::O

14) −e −7.0 −S 3 —H ::N

15) −e −7.0 −S 3 —H ::O

16) −e −7.0 −S 4 —H ::N

17) −e −7.0 −S 4 —H ::O

For the results with grid box 1, filters 12 and 13 each harvested 70 and 51 compounds, respectively. Those filtered sets were pooled together to form a set of 96 unique compounds for visual inspection. Filters 14 (which harvested 11 compounds), 15 (which harvested 21 compounds), and 1 (which harvested 34 compounds) were pooled together from the results with grid box 2, in order to identify 52 compounds for visual inspection. Similarly, for the results with grid box 3, filters 1 (which harvested 25 compounds), 14 (which identified 20 candidates), and 15 (which harvested 13 compounds) were pooled to obtain 34 compounds for visual inspection. To identify candidates in the results with grid box 4, filters 1 (which harvested 26 compounds), 14 (which harvested 19 compounds), and 15 (which harvested 14 compounds) were pooled to obtain 42 compounds. These four different pools of potentially promising compounds were then visually inspected to select the ligands to be tested experimentally for binding to recombinant E2 protein.

### Example 3

### Ligands Predicted to Bind to CD81 Binding Sites on E2

Five ligand-binding sites on the HCV E2 homology model (FIG. 2-6) were identified by blind docking of the Diversity Set III library of ligands to the E2 model. Each of these sites is associate with or positioned next to one or more of the amino acid or peptide sequences that have been identified by others to either participate in E2 binding to CD81, to E1 or to be important for HCV infectivity. The first sequence of importance is the peptide segment Q412-N423 that was identified to bind to the broadly neutralizing antibody AP33 [13, 42]. Alanine mutagenesis studies have shown all of the amino acids in this region appear to be important for HCV infectivity [43]. The model used in this study currently contains only three of the amino acids that correspond to this site, H421, 1422 and N423. Sequence 2 spans the second hyper-variable domain of E2, extending from amino acid Y474 to R492 [13, 21, 43-45]. The majority of amino acids in this sequence have been shown to have no effect on E2 binding to CD81 when mutated, but antibodies binding to this region of the protein do inhibit HCV infectivity [45] and CD81 binding [46]. One amino acid located within this domain, W487, does however appear to be critical for E2 binding to E1. This amino acid is the first residue in one of the WHY motifs that have been reported to play a role in E1:E2 dimerization [47]. The third sequence spans amino acids S522-G551 [13,21,43-45] and the fourth sequence of importance is comprised of amino acids P612-P619 [21,44]. Mutations of residues Y527, W529, D535, Y613, R614, W616, H617 and Y618 in these two regions have all been shown to eliminate E2 binding to CD81. Mutating all but three of these amino acids (D535, R614 and W616) appears to eliminate specific interactions with CD81. W616 is the first amino acid in another WHY motif that is located in a region (G600-C620) that has been shown to be involved in fusion [48]. Alanine mutagenesis of D535, R614 and W616 was found to disrupt the structure of the AR3A epitope and indirectly impact CD81 binding.

These five binding sites were used to guide to our selection of the top virtual screening hits to be tested experimentally for binding to recombinant E2 protein. While there is still some debate regarding the importance of the entire domains bound by neutralizing antibodies, amino acid mutagenesis studies have provided a great deal of insight into those amino acids located within the epitopes that participate in E2 binding to CD81. Based on this information, we have used the set of amino acids W420-1422, S424, G523, Y527, W529, G530, D535, P612-R614 and W616-P619 whose mutation has been shown to eliminate E2 binding to CD81 to identify locations within these five sites (FIGS. 2-6) where ligand binding would be expected to disrupt E2's ability to bind to CD81.

Blind docking of the 1,715 small molecules in the NCI Diversity III ligand set to the model of E2 led to the identification of a group of 34 ligands that were predicted to bind to one or more of these five sites (Table 1). The best ligands were considered to be those that exhibited the lowest free energy of binding and were predicted to interact with or bind nearby one or more of the E2 amino acids within the sites that were reported to be critical for E2 binding to CD81. The free energy of binding predicted for the best bound ligand conformations, shown in Table 1, ranged from −3.9 to −8.7 Kcal/mol Å³. Additional criteria used to select among the group of ligands predicted to bind include the number of contact points/interactions (such as hydrogen bonds, salt bridges, Van Der Waals interactions) with amino acids in the model (the larger number of contacts or interactions the better) and the chemical structure of the ligands (preference is given to those that contain a free amino or carboxyl group that is exposed to solvent). Compounds that have been shown previously to be highly toxic were excluded.

TABLE 1
Ligands predicted to bind to the HCV E2 protein by
blind docking of the NCI Diversity set III small
molecule library to the HCV E2 structural model.

| | Free | | Free |
|---|---|---|---|
| | Binding | Ligand | Binding |
| Ligand | Energy | ID | Energy |
| ID NCI | (Kcal/mol | NCI | (Kcal/mol |
| Number | Å³) | Number | Å³) |
| 670283 | −7.69 | 211490 | −8.7 |
| 86467 | −7.47 | 113486 | −6.26 |
| 639174 | −7.81 | 144694 | −7.27 |
| 81462 | −6.81 | 4429 | −7.3 |
| 403379 | −7.58 | 133071 | −7.5 |
| 213700 | −7.89 | 163910 | −7.4 |
| 359472 | −7.91 | 54709 | −7.3 |
| 146554 | −7.67 | 135618 | −8.7 |
| 204232 | −8.54 | 281254 | −6.5 |
| 281816 | −8.64 | 319990 | −7.4 |
| 308835 | −8.4 | 369070 | −6.3 |
| 60785 | −7.48 | 59620 | −7.3 |
| 84100 | −6.99 | 38968 | −3.9 |
| 158413 | −7.9 | 171303 | −5.8 |
| 57103 | −6.36 | 228155 | −8.7 |
| 121861 | −8.16 | 13316 | −6.8 |
| 3076 | −7.71 | 117268 | −7.6 |

### Example 4

### Experimental Analysis of Ligand Binding to Recombinant E2

A. Expression and Purification of the HCV E2 Protein Con1eE2

A construct containing the sequence encoding amino acids 384-656 of the Con1 envelope protein 2 ectodomain (eE2) [31], a genoptype 1 E2 sequence, was cloned into a lentiviral expression vector with a carboxy-terminal Protein A tag separated by a PreScission Protease cleavage consensus sequence. eE2-ProtA was stably expressed in HEK293T cells using lentiviral infection. The protein was secreted into the media and supernatants were purified using IgG Sepharose (GE Healthcare, Piscataway, N.J.). eE2-ProtA was eluted with 100 mM sodium citrate and 20 mM KCl at pH 3 directly into tubes containing 1M Tris pH 9 for immediate neutralization. PreScission Protease was added to the eluted sample at a ratio of 1:50 (enzyme:eE2), and the digest was then dialyzed into 20 mM HEPES pH 7.5, 250 mM NaCl, 5% glycerol. eE2 was separated from the cleaved tag and the PreScission Protease by ion exchange chromatography [32].

B. Experimental Analysis of Ligand Binding to Recombinant E2 by Surface Plasmon Resonance (SPR)

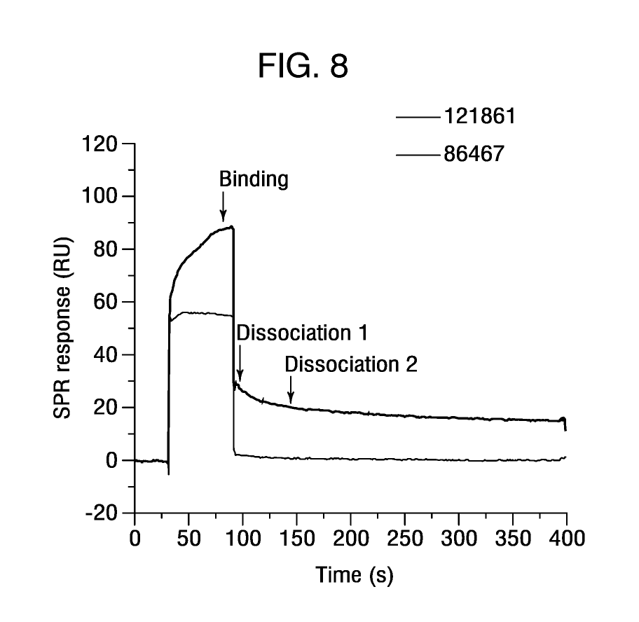

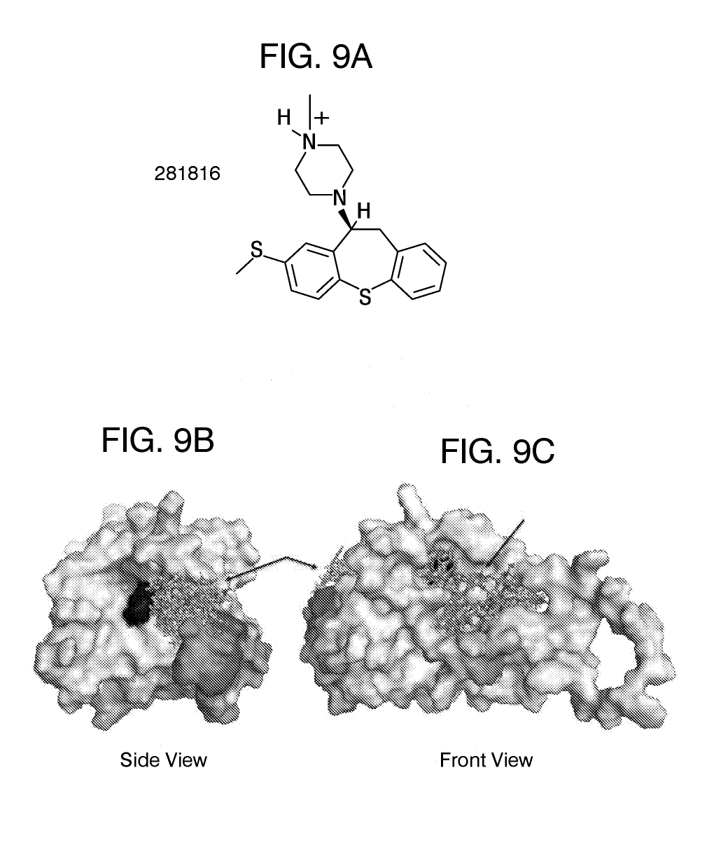

The set of 34 of the ligands predicted by AutoDock to bind to E2 were tested experimentally to determine if they bind to recombinant E2 protein immobilized on a chip using surface Plasmon resonance. The SPR analyses were performed using a Biacore T100 workstation (GE Healthcare, NJ, USA) and Prot-A-tagged HCV E2 protein. 1 μM ProtA-HCV E2 was diluted into 10 mM sodium acetate buffer pH 5 and immobilized for 15 min at a flow speed of 5 μl/min onto a CM5 sensor chip using amine coupling (EDC-NHS). Approximately 10,000 response units (RU) of protein were immobilized on the chip. His-CD81-LEL (Bioclone-CA/USA) binding to HCV E2 was tested prior to injecting the ligands to confirm the E2 protein was functional and would bind CD81-LEL. In a typical experiment with CD81, 411 of his-CD81 in 114 μl PBS was injected into channel 2 and 106.4 RUs of CD81 bound to the E2 on the chip. This was followed by testing the binding of the 34 virtual screening hits where the ligands were prepared as 200 μM solutions in PBS and they were introduced to the protein using a pre-programmed 3 min association and 1 min dissociation interval. The response was measured at two time points during dissociation, 10 and 50 seconds, to obtain information on the rate of ligand dissociation from E2. Twenty-three of the ligands predicted by AutoDock to bind to E2 were observed by SPR to bind to the recombinant protein (Table 2). The measured responses for the ligands that bound varied from 54 to 276 RUs. Data was also obtained on the rate of ligand dissociation by measuring the amount of ligand remaining bound at two time points, dissociation 1 (10 seconds) and dissociation 2 (50 seconds), during the rinsing of the chip with buffer (FIG. 8). The majority of the ligands dissociated quickly, as one might expect for small molecules that bind to the surface of a protein. A few, such as ligands 121861, 4429, 158413, 81462, and 57103, exhibited slower off rates when compared to others.

TABLE 2
Magnitude of surface Plasmon resonance binding response obtained
for the 23 ligands that were identified to bind to recombinant
E2 protein immobilized on a CM5 sensor chip. The rate of
ligand dissociation is assessed by measuring the response
units at two time points (10 sec and 50 sec) after the chip
with bound ligand is rinsed with buffer.

| Ligand | | | |
|---|---|---|---|
| ID NCI | Binding | Dissociation | Dissociation |
| Number | (RU) | 1 (RU) | 2 (RU) |
| 670283 | 54.3 | 4 | 1.4 |
| 86467 | 54.9 | 1.9 | 0.8 |
| 639174 | 55.4 | 2.3 | 0.6 |
| 81462 | 57.2 | 9.2 | 6.5 |
| 403379 | 58 | 2.8 | 1.1 |
| 213700 | 62 | 3.1 | 0.8 |
| 359472 | 62 | 2.5 | 0.8 |
| 146554 | 63.4 | 3.1 | 0.8 |
| 204232 | 63.4 | 2.5 | 0.4 |
| 281816 | 64.5 | 3.7 | 0.9 |
| 308835 | 64.8 | 7.1 | 5.2 |
| 60785 | 70.4 | 2.8 | 0.6 |
| 84100 | 71.2 | 4.2 | 2.2 |
| 158413 | 71.2 | 10.3 | 8.5 |
| 57103 | 81.6 | 11.4 | 2.5 |
| 121861 | 88.4 | 26.1 | 20.4 |
| 117268 | 88.5 | 4.1 | 1.2 |
| 3076 | 92.2 | 3.2 | 1.6 |
| 211490 | 102.9 | 6.1 | 2.1 |
| 113486 | 104.7 | 7 | 2.6 |
| 144694 | 118.8 | 6 | 2.3 |
| 4429 | 155.3 | 28.9 | 14.2 |
| 133071 | 276.3 | 1.8 | −2 |

Each of these ligands was predicted to bind to one or more of the five sites on E2 that contained or were immediately adjacent to peptide segments or amino acids that have been shown previously by others to be involved in binding to CD81, forming a dimer with HCV E1, or required for HCV infectivity (Table 3).

TABLE 3
Each of the 23 ligands identified by SPR to bind to recombinant
HCV E2 protein bound to a site that participates in CD81 binding,
E1:E2 dimerization or is required for HCV infectivity. Ligand
numbers correspond to those used in the NCI Diversity Set.

| | Target Sites on E2 Involved In | | |
|---|---|---|---|
| Ligand | CD81 Binding | E1 Binding | Other |
| 281816 | X | X | |
| 3076 | X | X | X |
| 4429 | X | X | |
| 57103 | X | X | X |
| 60785 | X | X | |
| 81642 | X | X | |
| 84100 | X | X | |
| 86467 | X | X | |
| 113486 | X | X | |
| 117268 | X | X | X |
| 121861 | X | X | X |
| 133071 | X | X | |
| 144694 | X | X | |
| 146554 | X | X | |
| 158413 | X | X | |
| 204232 | X | X | |
| 211490 | X | X | |
| 213700 | X | X | X |
| 308835 | X | X | |
| 359472 | X | X | |
| 403379 | X | X | |
| 639174 | X | X | X |
| 670283 | X | X | |

### Example 5

### Confirmation that Ligands Inhibit Infection by HCV

A. HCV Infection Assays

Pseudotyped retroviral particles harboring HCV envelope proteins (HCVpp) from different genotypes were produced as described previously [33, 34]. A plasmid encoding the feline endogenous virus RD114 glycoprotein [35] was used for the production of RD114pp. Both HCVpp and RD114pp expressed Firefly luciferase.

The cell culture-produced HCV particles (HCVcc) used in this study were based on the JFH1 strain [36] and were prepared as described previously [37, 38]. They were engineered to express the A4 epitope, titer-enhancing mutations and Gaussia luciferase [38,39].

To identify ligands that inhibit HCV infection, Huh-7 cells were seeded in 96-well plates and treated the day after with six different concentrations of each ligand diluted in DMSO in duplicate using a Zephyr automated liquid handling workstation (Caliper BioSciences, Hopkinton, Mass.). The final concentration of DMSO (1%) was adjusted to be the same for all ligand concentrations. Cells treated with DMSO were used as negative controls. Cells treated with different concentrations of anti-CD81 (JS-81 from BD Pharmingen, San Jose, Calif.) 1 hour before infection, were also used as positive controls. The third day, RD114pp, HCVpp or HCVcc were inoculated and incubated for 30 hours at 37° C. Firefly and Gaussia luciferase assays were performed as indicated by the manufacturer (Promega, San Luis Obispo, Calif.).

The analysis of the effect of 281816 ligand on Huh-7 infection by HCVpp bearing envelope proteins from different genotypes was performed in 24-well plates using the method described above. This ligand was also screened for toxicity to the hepatocytes using the MTS (3-(4,5-dimethylthiazol-2-yl)-5-(3-carboxymethoxyphenyl)-2-(4-sulfophenyl)-2Htetrazolium) assay [40].

B. Inhibition of HCV Infection

The twenty-three compounds that were observed to bind to recombinant E2 protein were then tested to determine if they would block hepatocyte infection by HVCpp and HVCcc virus particles. Pseudotyped retroviral particles harboring the envelope protein of an endogenous feline retrovirus (RD114pp) were first used to determine the specificity and the safety of molecules. We excluded from a further characterization the molecules for which the half maximal inhibitory concentration (IC50) against RD114pp was greater than 10 μM or the molecules that significantly increased RD114pp infection (Table 4).

TABLE 4
The IC50 values obtained for the 23 ligands screened for their ability
to inhibit HCVcc, HCVpp and RD114pp infection of Huh-7 cells. The
IC50 is the concentration required to block infection by 50%.

| Ligand | IC50 (μM) | | |
|---|---|---|---|
| NCI # | RD114pp | HCVpp | HCVcc |
| 670283 | 3 | ND | ND |
| 86467 | >10 | >10 | >10 |
| 639174 | 0.03 | ND | ND |
| 81462 | >10 | >10 | >10 |
| 403379 | >10 | >10 | >10 |
| 213700 | >10 | >10 | >10 |
| 359472 | >10 | >10 | >10 |
| 146554 | >10 | ND | ND |
| 204232 | >10 | >10 | >10 |
| 281816 | >10 | 1.02 | 3.95 |
| 308835 | >10 | >10 | >10 |
| 60785 | 3.5 | ND | ND |
| 84100 | >10 | >10 | >10 |
| 158413 | >10 | >10 | >10 |
| 57103 | 0.3 | ND | ND |
| 121861 | >10 | >10 | >10 |
| 117268 | 0.1 | >10 | >10 |
| 3076 | 0.25 | ND | ND |
| 211490 | 0.5 | ND | ND |
| 113486 | >10 | >10 | >10 |
| 144694 | >10 | >10 | >10 |
| 4429 | >10 | >10 | >10 |
| 133071 | 0.10 | ND | ND |
| Anti- | >10 | 0.17 | 0.36 |
| CD81 | | | |

ND: not determined

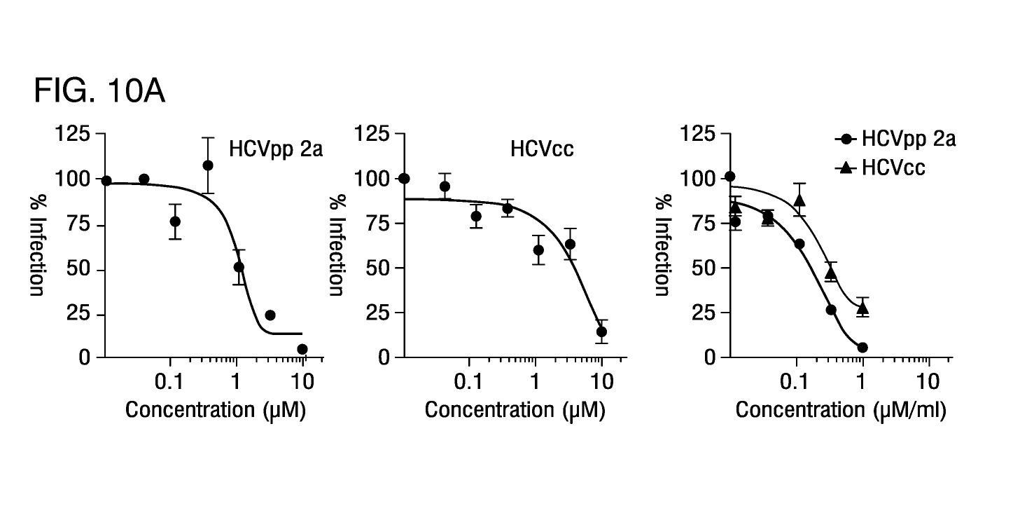

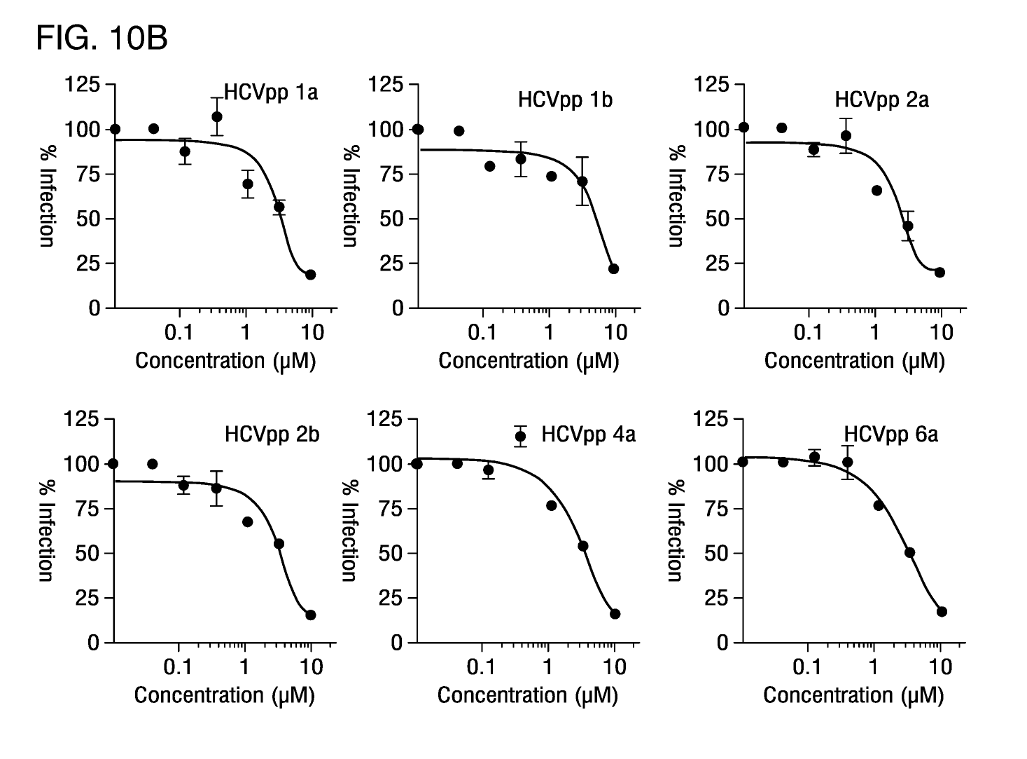

The remaining ligands were next tested against pseudotyped retroviral particles harboring genotype-2a HCV envelope proteins (HCVpp 2a), cell culture produced HCV particles (HCVcc) or RD114pp. As a positive control, an anti-CD81 antibody was used in parallel. One compound (281816) showed an inhibitory effect on HCVpp and HCVcc infection with IC50 of 1.02 μM and 3.95 μM, respectively (Table 4 and FIG. 10A), indicating that this molecule inhibits the entry step of the HCV lifecycle, probably through a specific effect on the virus's interaction with CD81. While hepatocyte toxicity was not observed over the range of ligand 281816 concentrations tested in the assays (the highest concentration tested was 10 μM), a subsequent viability assay showed a 50% cytotoxic concentration (CC50) for 281816 of 14 μM, as determined using an MTS assay (data not shown).

### Example 6

### Confirmation that Ligand 281816 Inhibits Infection by Other HCV Subtypes

To determine if 281816 would inhibit HCV genotypes other than 2a, a series of infection assays were performed with HCVpp bearing envelope proteins from a number of different HCV genotypes. Interestingly, 281816 was found to be equally effective in inhibiting hepatocyte infection by all the HCV genotypes tested (1a, 1b, 2a, 2b, 4a and 6a, FIG. 10). The IC50 values ranged from 2.2 μM to 4.6 μM (Table 5).

TABLE 5
Genotype independent inhibition of HCVpp
infection of Huh-7 cells by ligand 281816

| Subtypes | IC50 (μM) |
|---|---|
| HCVpp 1a | 2.95 |
| HCVpp 1b | 4.66 |
| HCVpp 2a | 2.22 |
| HCVpp 2b | 2.93 |
| HCVpp 4a | 3.44 |
| HCVpp 6a | 3.30 |

### Example 7

### Inhibition of Recombinant E2 Binding to Native CD81

A. Blocking of Binding of HCV E2 to CD81.

The human B cell line, Raji, which expresses high levels of CD81 on its surface was used to determine if ligands could inhibit the binding of HCV-E2 protein to native CD81. Cells were grown in RPMI medium (10% fetal calf serum, 1% penicillin/streptomycin, 1% L-glutamine, 1% non-essential amino acids, 1% sodium pyruvate, pH 7.4) at 37° C. in an atmosphere of 5% CO₂. Purified HCV-E2 protein (4 μg) was pre-incubated with 1,5,15, 50, 100 or 400 μM of the ligand 281816 for 25 min RT. After pre-incubation the E2-ligand complex was added to the cells and incubated for 25 min. The complexes were washed from the cells and 0.5 μg of anti E2 antibody (clone H53) was added followed by secondary anti mouse-FITC (Southern Biotechnology). The cells were washed, fixed with 3% paraformaldehyde, and analyzed by flow cytometry (BD FACSCalibur, software: Cell Quest Pro) analysis. The mean fluorescence intensity (MFI) was calculated using Flowjo software (TreesStar, www.flowjo.com).

A. Blocking of E2 Binding to CD81 by Ligand 281816

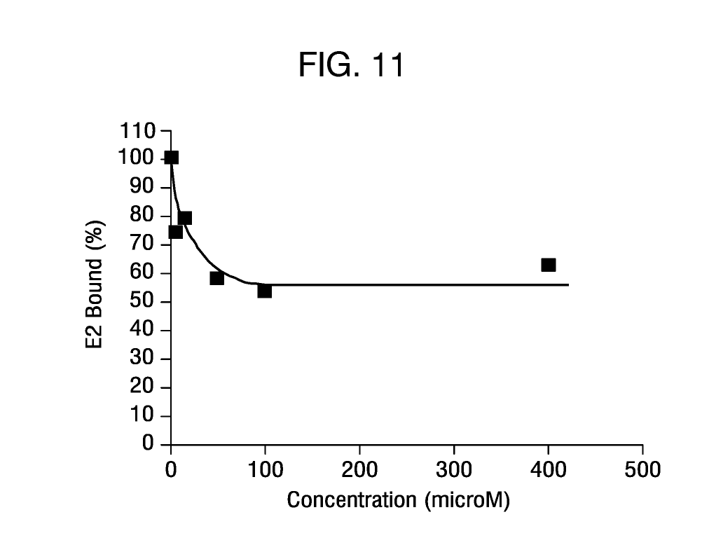

Ligand 281816 was selected based on the prediction by docking that it would bind to a site on the HCV E2 protein where CD81 binds. To confirm that the binding of 281816 to E2 inhibits the HCV E2-CD81 interaction, flow cytometry was used to monitor the binding of a recombinant form of the E2 protein to native CD81 overexpressed on Raji cells as a function of 281816 concentration. As shown by FIG. 11, binding of the E2 protein to Raji cells is inhibited by 281816 in a dose dependent manner.

### REFERENCES

1. Anwar, M. I., Rahman, M., Hassan, M. U. & Iqbal, M. (2013) Prevalence of active hepatitis C virus infections among general public of Lahore, Pakistan, Virology J 10: 351
2. Blackard, J. T., Shata, M. T., Shire, N.J. & Sherman, K. E. (2008) Acute hepatitis C virus infection: a chronic problem, Hepatology 47: 321.
3. Zeuzem, S., Berg, T., Moeller, B., Hinrichsen, H., Mauss, S., Wedemeyer, H., Sarrazin, C., Hueppe, D., Zehnter, E. & Manns, M. P. (2009) Expert opinion on the treatment of patients with chronic hepatitis C, J Viral Hepatitis 16: 75.
4. Marks, K. M. & Jacobson, I. M. (2012) The first wave: HCV NS3 protease inhibitors telaprevir and boceprevir, Antiviral Therapy 17: 1119.
5. Colombo, M., Fernandez, I., Abdurakhmanov, D., Ferreira, P. A., Strasser, S. I., Urbanek, P., Moreno, C., Streinu-Cercel, A., Verheyen, A., Iraqi, W., Demasi, R., Hill, A., Lauffer, J. M., Lonjon-Domanec, I. & Wedemeyer, H. (2013) Safety and on-treatment efficacy of telaprevir: the early access programme for patients with advanced hepatitis C, Gut 0: 1.
6. Asselah, T. (2014) Sofosbuvir for the treatment of hepatitis C virus, Expert Opinion on Pharmacotherapy 15: 121.
7. Lenz, O., Vijgen, L., Berke, J. M., Cummings, M. D., Fevery, B., Peeters, M., De Smedt, G., Moreno, C. & Picchio, G. (2013) Virologic response and characterisation of HCV genotype 2-6 in patients receiving TMC435 monotherapy (study TMC435-C202), J Hepatology 58: 445.
8. El-Awady, M. K., Tabll, A. A., El-Abd, Y. S., Yousif, H., Hegab, M., Reda, M., El Shenawy, R., Moustafa, R. I., Degheidy, N. & El Din, N. G. (2009) Conserved peptides within the E2 region of Hepatitis C virus induce humoral and cellular responses in goats, Virology J 6: 66.
9. Carlsen, T. H., Scheel, T. K., Ramirez, S., Foung, S. K. & Bukh, J. (2013) Characterization of hepatitis C virus recombinants with chimeric E1/E2 envelope proteins and identification of single amino acids in the E2 stem region important for entry, J Virology 87: 1385.
10. Li, Y. P., Kang, H. N., Babiuk, L. A. & Liu, Q. (2006) Elicitation of strong immune responses by a DNA vaccine expressing a secreted form of hepatitis C virus envelope protein E2 in murine and porcine animal models, World Journal of Gastroenterology 12: 7126.
11. Ray, R., Meyer, K., Banerjee, A., Basu, A., Coates, S., Abrignani, S., Houghton, M., Frey, S. E. & Belshe, R. B. (2010) Characterization of antibodies induced by vaccination with hepatitis C virus envelope glycoproteins, J Infectious Diseases 202: 862.
12. Kong, L., Giang, E., Nieusma, T., Kadam, R. U., Cogburn, K. E., Hua, Y., Dai, X., Stanfield, R. L., Burton, D. R., Ward, A. B., Wilson, I. A. & Law, M. (2013) Hepatitis C virus E2 envelope glycoprotein core structure, Science 342: 1090.
13. Kong, L., Giang, E., Nieusma, T., Robbins, J. B., Deller, M. C., Stanfield, R. L., Wilson, I. A. & Law, M. (2012) Structure of hepatitis C virus envelope glycoprotein E2 antigenic site 412 to 423 in complex with antibody AP33, J Virology 86: 13085.
14. Law, M., Maruyama, T., Lewis, J., Giang, E., Tarr, A. W., Stamataki, Z., Gastaminza, P., Chisari, F. V., Jones, I. M., Fox, R. I., Ball, J. K., McKeating, J. A., Kneteman, N. M. & Burton, D. R. (2008) Broadly neutralizing antibodies protect against hepatitis C virus quasispecies challenge, Nature Medicine 14: 25.
15. Pileri, P., Uematsu, Y., Campagnoli, S., Galli, G., Falugi, F., Petracca, R., Weiner, A. J., Houghton, M., Rosa, D., Grandi, G. and Abrignani, S. (1998) Binding of hepatitis C virus to CD81, Science 282: 938.
16. Petracca, R., Falugi, F., Galli, G., Norais, N., Rosa, D., Campagnoli, S., Burgio, V., Di Stasio, E., Giardina, B., Houghton, M., Abrignani, S. and Grandi, G. (2000) Structure-function analysis of hepatitis C virus envelope-CD81 binding, J Virology 74: 4824.
17. Higginbottom, A., Quinn, E. R., Kuo, C. C., Flint, M., Wilson, L. H., Bianchi, E., Nicosia, A., Monk, P. N., McKeating, J. A. and Levy, S. (2000) Identification of amino acid residues in CD81 critical for interaction with hepatitis C virus envelope glycoprotein E2, J Virology 74, 3642.
18. Drummer, H. E., Wilson, K. A. and Poumbourios, P. (2002) Identification of the hepatitis C virus E2 glycoprotein binding site on the large extracellular loop of CD81, J Virology 76: 11143.
19. Zhang, Y. Y., Zhang, B. H., Ishii, K. and Liang, T. J. (2010) Novel function of CD81 in controlling hepatitis C virus replication, J Virology 84: 3396.
20. Ahlenstiel, G. (2013) The Natural Killer Cell Response to HCV Infection, Immune Network 13: 168.
21. Zemla, A., Zhou, C. E., Slezak, T., Kuczmarski, T., Rama, D., Torres, C., Sawicka, D., Barsky, D. (2005) AS2TS system for protein structure modeling and analysis, Nucleic Acids Res 33: W111-115.
22. Zemla, A. T., Lang, D. M., Kostova, T., Andino, R., Ecale Zhou, C. L. (2011) StralSV: assessment of sequence variability within similar 3D structures and application to polio RNA-dependent RNA polymerase, BMC Bioinformatics 12:226.
23. Krivov, G. G., Shapovalov, M. V., and Dunbrack Jr, R. L. (2009) Improved prediction of protein side-chain conformations with scwrl4. Proteins 77: 778.
24. Chen, V. B., Arendall 3^{rd}, W. B., Headd, J. J., Keedy, D. A., Immormino, R. M., Kapral, G. J., Murray, L. W., Richardson, J. S., Richardson, D. C. (2010) MolProbity: all-atom structure validation for macromolecular crystallography, Acta Crystallogr D Biol Crystallogr 66:12.
25. Pettersen, E. F., Goddard, T. D., Huang, C. C., Couch, G. S., Greenblatt, D. M., Meng, E. C., and Ferrin, T. E. (2004) UCSF chimera—a visualization system for exploratory research and analysis, J Comput Chem 25: 1605.
26. Trott, O. & Olson, A. J. (2010) AutoDock Vina: improving the speed and accuracy of docking with a new scoring function, efficient optimization and multithreading, J Comput Chem 31: 455.
27. Chen, V. B., Arendall, W. B., Headd, J. J. Keedy, D. A., Immormino, R. M., Kapral, G. J., Murray, L. W., Richardson, J. S. & Richardson, D. C. (2010) MolProbity: All-atom structure validation for macromolecular crystallography, Acta Cryst D66: 12.
28. Morris G. M., Huey R., Lindstrom W., Sanner M. F., Belew R. K., Goodsell D. S. & Olson A. J. (2009) AutoDock4 and AutoDockTools4: Automated docking with selective receptor flexibility. J Comput Chem 30: 2785.
29. Irwin, J. J., Sterling, T., Mysinger, M. M., Bolstad, E. S. & Coleman, R. G. (2012) ZINC: A free tool to discover chemistry for biology. J Chem Inf Model 52: 1757.
30. Forli, S. Raccoon <http://autodock.scripps.edu/resources/raccoon>. Accessed 2013. Molecular Graphics Laboratory, The Scripps Research Institute, La Jolla, Calif., 2010.
31. Bianchi, A., Crotta, S., Brazzoli, M., Foung, S. K. & Merola, M. 2011, “Hepatitis C virus e2 protein ectodomain is essential for assembly of infectious virions”, International journal of hepatology, vol. 2011, pp. 968161.
32. http://www.gelifesciences.com/webapp/wcs/stores/servlet/productById/en/GELifeSciences/27084301
33. Bartosch, B., Bukh, J., Meunier, J. C., Granier, C., Engle, R. E., Blackwelder, W. C., Emerson, S. U., Cosset, F. L. & Purcell, R. H. (2003) In vitro assay for neutralizing antibody to hepatitis C virus: evidence for broadly conserved neutralization epitopes, Proc Nat Acad Sci U. S 100: 14199.
34. Op De Beeck, A., Voisset, C., Bartosch, B., Ciczora, Y., Cocquerel, L., Keck, Z., Foung, S., Cosset, F. L. & Dubuisson, J. (2004) Characterization of functional hepatitis C virus envelope glycoproteins, J Virology 78: 2994.
35. Sandrin, V., Boson, B., Salmon, P., Gay, W., Negre, D., Le Grand, R., Trono, D. & Cosset, F. L. (2002) Lentiviral vectors pseudotyped with a modified RD114 envelope glycoprotein show increased stability in sera and augmented transduction of primary lymphocytes and CD34+ cells derived from human and nonhuman primates, Blood 100: 823.
36. Wakita, T., Pietschmann, T., Kato, T., Date, T., Miyamoto, M., Zhao, Z., Murthy, K., Habermann, A., Krausslich, H. G., Mizokami, M., Bartenschlager, R. & Liang, T. J. (2005) Production of infectious hepatitis C virus in tissue culture from a cloned viral genome, Nature Medicine 11: 791.
37. Rocha-Perugini, V., Montpellier, C., Delgrange, D., Wychowski, C., Helle, F., Pillez, A., Drobecq, H., Le Naour, F., Charrin, S., Levy, S., Rubinstein, E., Dubuisson, J. & Cocquerel, L. (2008) The CD81 partner EWI-2wint inhibits hepatitis C virus entry, PloS One 3: e1866.
38. Delgrange, D., Pillez, A., Castelain, S., Cocquerel, L., Rouille, Y., Dubuisson, J., Wakita, T., Duverlie, G. & Wychowski, C. (2007) Robust production of infectious viral particles in Huh-7 cells by introducing mutations in hepatitis C virus structural proteins, J Gen Virology 88: 2495.
39. Goueslain, L., Alsaleh, K., Horellou, P., Roingeard, P., Descamps, V., Duverlie, G., Ciczora, Y., Wychowski, C., Dubuisson, J. & Rouille, Y. (2010) Identification of GBF1 as a cellular factor required for hepatitis C virus RNA replication, J Virology 84: 773.
40. Malich, G., Markovic, B. & Winder, C. (1997) The sensitivity and specificity of the MTS tetrazolium assay for detecting the in vitro cytotoxicity of 20 chemicals using human cell lines, Toxicology 124: 179.
41. Zemla, A. (2003) LGA—A method for finding 3d similarities in protein structures, Nucl Acids Res 31: 3370.
42. Tarr, A. W., Owsianka, A. M., Timms, J. M., McClure, C. P., Brown, R. J., Hickling, T. P., Pietschmann, T., Bartenschlager, R., Patel, A. H. & Ball, J. K. (2006) Characterization of the hepatitis C virus E2 epitope defined by the broadly neutralizing monoclonal antibody AP33, Hepatology 43: 592.
43. Owsianka, A. M., Timms, J. M., Tarr, A. W., Brown, R. J., Hickling, T. P., Szwejk, A., Bienkowska-Szewczyk, K., Thomson, B. J., Patel, A. H. & Ball, J. K. (2006) Identification of conserved residues in the E2 envelope glycoprotein of the hepatitis C virus that are critical for CD81 binding, J Virology 80: 8695.
44. Roccasecca, R., Ansuini, H., Vitelli, A., Meola, A., Scarselli, E., Acali, S., Pezzanera, M., Ercole, B. B., McKeating, J., Yagnik, A., Lahm, A., Tramontano, A., Cortese, R. & Nicosia, A. (2003) Binding of the hepatitis C virus E2 glycoprotein to CD81 is strain specific and is modulated by a complex interplay between hypervariable regions 1 and 2, J Virology 77:1856.
45. Rothwangl, K. B., Manicassamy, B., Uprichard, S. L. & Rong, L. (2008) Dissecting the role of putative CD81 binding regions of E2 in mediating HCV entry: putative CD81 binding region 1 is not involved in CD81 binding, Virology J 5: 46.
46. Flint, M., Maidens, C., Loomis-Price, L. D., Shotton, C., Dubuisson, J., Monk, P., Higginbottom, A., Levy, S. & McKeating, J. A. (1999) Characterization of hepatitis C virus E2 glycoprotein interaction with a putative cellular receptor, CD81. J Virology 73: 6235.
47. Yi, M., Nakamoto, Y., Kaneko, S., Yamashita, T., Murakami, S. (1997) Delineation of regions important for heteromeric association of hepatitis C virus E1 and E2. Virology 231:119.
48. Lavillette, D., Pecheur, E. I., Donot, P., Fresquet, J., Molle, J., Corbau, R., Dreux, M., Penin, F. & Cosset, F. L. (2007) Characterization of fusion determinants points to the involvement of three discrete regions of both E1 and E2 glycoproteins in the membrane fusion process of hepatitis C virus, J Virology 81:8752.

1. A conjugate that binds to Hepatitis C Virus (HCV) E2 protein comprising at least two small molecule ligands that bind to HCV E2 protein,
wherein at least one of said two small molecule ligands is 281816 covalently bound to at least one small organic molecule ligand that binds to HCV E2 selected from the group consisting of 146554, 3076, 4429, 57103, 60785, 81462, 84100, 86467, 113486, 117268, 121861, 133071, 144694, 158413, 171303, 204232, 211490, 213700, 215629, 281254, 281816, 308835, 359472, 403379, 639174, and 6702831; or
wherein the at least two small molecule ligands are selected from the group consisting of 3076, 4429, 57103, 60785, 81462, 84100, 86467, 113486, 117268, 121861, 133071, 144694, 146554, 158413, 171303, 204232, 211490, 213700, 215629, 281254, 281816, 308835, 359472, 403379, 639174, and 6702831; and, optionally,
wherein the at least two small molecule ligands may be linked together via a linker or carrier.

2. The conjugate of claim 1, wherein at least one of said two small organic molecule ligands is 281816 covalently bound to at least one small organic molecule ligand that binds to HCV E2 selected from the group consisting of 3076, 4429, 57103, 60785, 81462, 84100, 86467, 113486, 117268, 121861, 133071, 144694, 146554, 158413, 171303, 204232, 211490, 213700, 215629, 281254, 281816, 308835, 359472, 403379, 639174, and 6702831.

3. The conjugate of claim 1, wherein the at least two small organic molecule ligands are selected from the group consisting of 3076, 4429, 57103, 60785, 81462, 84100, 86467, 113486, 117268, 121861, 133071, 144694, 146554, 158413, 171303, 204232, 211490, 213700, 215629, 281254, 281816, 308835, 359472, 403379, 639174, and 6702831.

4. The conjugate of claim 1 that comprises a small molecule ligand selected from the group consisting of 73735, 57103, and 133071.

5. The conjugate of claim 1 that binds to at least one of HCV E2 Sites 1, 2, 3, 4 or 5.

6. The conjugate of claim 1 that comprises core structure 1, 2, 3 or 4.

7. A homomultimer comprising at least two or three small molecule ligands linked together, optionally via a linker or carrier, wherein said small molecule ligands are selected from the group consisting of 3076, 4429, 57103, 60785, 81462, 84100, 86467, 113486, 117268, 121861, 133071, 144694, 146554, 158413, 171303, 204232, 211490, 213700, 215629, 281254, 281816, 308835, 359472, 403379, 639174, and 6702831.

8. The conjugate of claim 1, further comprising at least one molecule that binds to CD81.

9. The use of the conjugate according to claim 1 for the preparation of a medicament for preventing or treating HCV infection.

10. A composition comprising the conjugate according to claim 1 and a pharmaceutically acceptable carrier or excipient.

11. A method for preventing a subject at risk of HCV infection from being infected with HCV comprising administering the conjugate of claim 1 to a subject in need thereof in an amount effective to prevent HCV infection.

12. A method for treating a subject infected with HCV comprising administering the conjugate of claim 1 to a subject in need thereof in an amount effective to reduce the severity of HCV infection.

13. A method for inhibiting or blocking the attachment of HCV to a cell comprising contacting HCV with the conjugate of claim 1.

14. A small organic molecule that binds to HCV E2 protein or a multimer or conjugate comprising said small organic molecule.

15. The small organic molecule of claim 14 that binds to at least one of Sites 1, 2, 3, 4 or 5.

16. The small organic molecule of claim 14 that comprises core structure 1, 2, 3 or 4.

17. The small organic molecule of claim 14 selected from the group consisting of 3076, 4429, 57103, 60785, 81462, 84100, 86467, 113486, 117268, 121861, 133071, 144694, 146554, 158413, 171303, 204232, 211490, 213700, 215629, 281254, 281816, 308835, 359472, 403379, 639174, and 6702831; or a molecule that binds at a site surrounded by or adjacent to at least four, five, six, seven or more of the same amino acid residues on HCV E2 protein as said molecule.

18. The molecule of claim 14 selected from the group consisting of 281816, 73735, 57103, and 133071; or a molecule that binds at a site surrounded by or adjacent to at least four, five, six, seven or more of the same amino acid residues on HCV E2 protein as said molecule.

19. The molecule of claim 14 that is 281816; or a molecule that binds at a site surrounded by or adjacent to at least four, five, six, seven or more of the same amino acid residues on HCV E2 protein as said molecule.

20. A method for preventing or treating an HCV infection comprising administering at least one molecule according to claim 14 to a subject in need thereof.
